(19) United States
(12) Patent Application Publication
(10) Pub. No.: US 2026/0048151 A1
(43) Pub. Date: Feb. 19, 2026

(54) HEMICYANINE PUSH-PULL DYES BASED ON CHROMENE ELECTRON DONORS

(71) Applicant: UNIVERSITY OF CONNECTICUT, Farmington, CT (US)

(72) Inventors: Leslie M. LOEW, Hartford, CT (US); Ping YAN, Avon, CT (US); Corey ACKER, West Hartford, CT (US)

(21) Appl. No.: 19/104,486

(22) PCT Filed: Aug. 22, 2023
(86) PCT No.: PCT/US2023/030865

§ 371 Date: Feb. 18, 2025

Related U.S. Application Data
(60) Provisional application No. 63/400,020, filed on Aug. 22, 2022.

(51) Int. Cl. A61K 49/00; C07D 311/58; C07D 405/06; C09B 23/04; C09K 11/02; C09K 11/06; G01R 19/155

(52) U.S. Cl. CPC A61K 49/0021; C07D 311/58; C07D 405/06; C09B 23/04; C09K 11/02; C09K 11/06; C09K 2211/1018; G01R 19/155

(57) ABSTRACT

The disclosed hemicyanine dyes contain an amino-chromene moiety as an electron donor, facilitating a variety of optical properties and uses in sensing applications.

### CROSS-REFERENCE TO RELATED APPLICATIONS

This application claims priority to U.S. Provisional Application No. 63/400,020, filed Aug. 22, 2022, the entirety of which is incorporated into this application by reference.

### BACKGROUND

Propelled by the need to image deep within living tissue, the past two decades have witnessed a burgeoning of research efforts in pushing fluorescent sensors into the near-infrared (NIR) window, 650-900 nm. where light scattering is diminished and where hemoglobin, oxyhemoglobin and water have minimal absorption. A standard method for seeking improvements is to extend conjugation length. For example, for symmetric cyanines containing a pair of indolenium heterocycles linked by a series of vinylenes, each additional double bond usually gives ca. 100 nm red shift. However, this comes with disadvantages such as lower solubility, lower fluorescence quantum efficiency, and lower photostability of the compounds enabling such sensors. In addition, extending the length of the vinylene linker chain does not produce such large bathochromic shifts for asymmetric chromophores where the terminal groups have very different electron affinities, as would be the case for push-pull chromophores. Push-pull chromophores have been successfully incorporated into optical sensors because their spectral properties are highly sensitive to their molecular environments.

Among the sensors that employ push-pull chromophores are voltage sensitive dyes (VSDs), which can report on the electrical state of cell membranes. VSDs have advantages over classical electrode-based measurements because they can be readily applied to camera-based imaging, recording activity simultaneously from millions of cells in a dish or within tissue. VSDs are also useful for subcellular imaging from regions that are not accessible to electrodes.

Research on VSDs of very different structural classes have also generated red absorbing VSDs, exemplified by an oxonol dye RH1691 and a silicon rhodamine BeRST1. All these VSDs allow the use of excitation around 630 nm in biological applications. The availability of VSDs still further to the red would enable deeper tissue imaging because the exciting light would be subject to less light scattering. Additionally, opening this higher spectral window would allow the VSDs to be used in combination with other fluorescent sensors (e.g., Ca₂₊, pH), multiple fluorescent protein labels, and optogenetic actuators. A need in the art exists for improvements to VSDs, particularly for biological sensing applications.

### SUMMARY

Embodiments of the compounds have the structure:

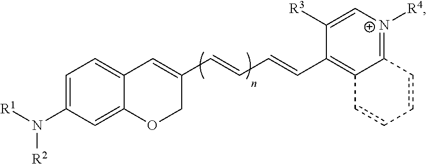

where R¹-R³ are independently hydrogen, halide, C₁-C₁₂ alkyl, C₁-C₁₂ heteroalkyl, C₂-C₁₂ alkenyl, C₂-C₁₂ alkynyl, C₁-C₁₂ haloalkyl, C₂-C₁₂ haloalkenyl, cycloalkyl, heterocycloalkyl, bicyclic cycloalkyl, bicyclic heterocycloalkyl, cycloalkenyl, cycloalkynyl, aryl, or heteroaryl; where n is an integer ranging from 0-4; where R⁴ is (C₁-C₁₂)-R₅, where R⁵ is NR⁶⁻⁸, where R⁶-R⁸ are independently C₁-C₁₂ alkyl; sulfonate; carboxylic acid; amino; —C(O)O—R⁹, where R⁹ is succinimidyl; or —NH—C(O)'R¹⁰—R¹¹, wherein R¹⁰ is C₁-C₄ alkyl and R¹¹ is maleimide; and wherein the dashed line (- - - -) denotes an optional ring structure.

Also described are methods for making the disclosed compounds, comprising reacting an aldehyde having the structure:

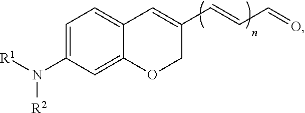

where R¹ and R² are independently hydrogen, halide, C₁-C₁₂ alkyl, C₁-C₁₂ heteroalkyl, C₂-C₁₂ alkenyl, C₂-C₁₂ alkynyl, C₁-C₁₂ haloalkyl, C₂-C₁₂ haloalkenyl, cycloalkyl, heterocycloalkyl, bicyclic cycloalkyl, bicyclic heterocycloalkyl, cycloalkenyl, cycloalkynyl, aryl, or heteroaryl; where n is an integer ranging from 0-4; with a pyridinium-based compound having the structure:

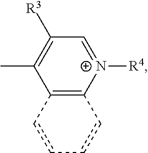

where R³ is hydrogen, halide, C₁-C₁₂ alkyl, C₁-C₁₂ heteroalkyl, C₂-C₁₂ alkenyl, C₂-C₁₂ alkynyl, C₁-C₁₂ haloalkyl, C₂-C₁₂ haloalkenyl, cycloalkyl, heterocycloalkyl, bicyclic cycloalkyl, bicyclic heterocycloalkyl, cycloalkenyl, cycloalkynyl, aryl, or heteroaryl; where R⁴ is (C₁-C₁₂)—R⁵, wherein R⁵ is NR⁶⁻⁸, wherein R₆—R⁸ are independently C₁-C₁₂ alkyl; sulfonate; carboxylic acid; amino; —C(O)O—R₉, wherein R⁹ is succinimidyl; or —NH—C(O)—R¹⁰—R¹¹, wherein R¹⁰ is C₁-C₄ alkyl and R¹¹ is maleimide; and where the dashed line (- - - -) denotes an optional ring structure.

Also described are kits comprising the compounds.

Also described are methods of recording ratiometric voltage with simultaneous optogenetic actuation with the use of a disclosed compound, e.g., through fluorescence imaging. Similarly, described are methods of imaging biological tissue with the use of described compound. Specific examples include in vivo cardiac imaging and blood-perfused imaging. Also described is a method of sensing a change in a biological environment with the use of a described compound.

Additional methods are also described, including methods of detecting a counterfeit product with the use of disclosed compound, e.g., by detecting one or more target analytes by measuring the fluorescence of a disclosed compound. Finally, optoelectronic devices can incorporate one or more disclosed compounds, e.g., as the light-emitter.

### BRIEF DESCRIPTION OF THE DRAWINGS

The drawings illustrate some alternative embodiments and assist in explaining the principles of the disclosure.

FIG. IA is a plot showing representative absorbance and emission spectral data for compound 2a in EtOH, PBS. and MLV.

FIG. 1B is a plot showing representative voltage sensitivity data for compound 2a as a function of excitation wavelength.

FIGS. 2A-2C are plots showing absorption and emission spectra of 2a, in comparison with ElectroFluor630, in various solvent environments: MLV, EtOH, and PBS. Multilamellar lipid vesicle suspension (MLV) was prepared by sonicating soybean phosphatidylcholine in PBS buffer (2 mg/mL) to mimic the cell membrane environment. The emission spectra in ethanol and PBS buffer were scaled 10 and 20 times respectively relative to the emission spectra in MLV. Excitation wavelength is 580 nm for emission spectra.

FIGS. 3A-3B are plots characterizing compound 2a in hemispherical lipid bilayer system. FIG. 3A: Fluorescence of 2a responses linearly to membrane voltage. Red: excitation wavelength=730 nm; Green: excitation wavelength=570 nm. Error bars are S.D. from 8 measurements at each point for 730 nm excitation, and 3 measurements for 570 nm excitation.

FIG. 3B: The voltage sensitivity depends critically on excitation wavelength. Red edge excitation is preferred for best sensitivity. Error bars indicate SD for 3 repeats except for 550 nm and 730 nm, which were 6 repeats. A 780 nm long pass filter was used to collect emission from all excitation wavelengths.

FIGS. 4A-4E show optical mapping of cardiac electrical activity in intact mouse hearts stained with ElectroFluor730p. FIG. 4A: Left, representative fluorescence images (FO) of a mouse heart stained with the ElectroFluor730p; excitation centered at 730 nm (39 nm width) and emission centered at 792 nm (64 nm width). Mouse hearts were electrically paced at the apex using an electrode with a burst of 15 stimuli at 10 Hz (the location of electrical stimulus is shown using a yellow bolt symbol). Right, four representative frames of the optical mapping (ΔF/F) recorded during the electrical stimulation. The electrical activation is reported in red and the baseline in green. FIG. 4B: Activation map of the mouse heart shown in A, showing the time for propagation of the fluorescently recorded AP along the surface of the heart. FIG. 4C: Fluorescent signals (ΔF/F) extracted from the red square in A showing 3 electrically-induced APs (APs were elicited corresponding to the yellow arrowheads). FIG. 4D: Optical AP parameters measured in mouse hearts stained with the VSD ElectroFluor730p and di-4-ANBDQPQ: AP amplitude: APA; AP rising slope: APRS; AP duration at 50. 70 and 90% of repolarization phase: APD50, APD70, APD90. Data was collected from 4 di-4-ANBDQPQ-stained mouse hearts and 3 ElectroFluor730p-stained mouse hearts. Each data point represents the average of AP parameters of a different heart. Also indicated is the mean and the standard error of the mean (SEM). For mean comparison. the student's t-test was applied showing no statistically significant differences in AP parameters excepting AP Amplitude (APA). FIG. 4E: Sensitivity analysis of ElectroFluor730p at three different excitation wavelengths: 530, 590, 730 nm. The 0% sensitivity of the dye (isosbestic point, ˜571 nm) was found by intercepting the exponential fit of the mean values with Y=0).

FIGS. 5A-D show recording of cardiac action potentials induced by optogenetic actuation of ChR2. FIG. 5A: ChR2 excitation spectrum (36) superimposed with ElectroFluor730p excitation and emission spectra. FIG. 5B and FIG. 5C: AP detection in mouse hearts stained with ElectroFluor730p during local optogenetic stimulation. Left: Representative fluorescence images (F0) of ChR2 (B) and CTRL (C) mouse hearts stained with the ElectroFluor730p. Optical stimulations (470 nm, 4.0 mW/mm2, 3 ms duration) were delivered at the apex (blue spot) of ChR2 and CTRL mouse hearts at a stimulation frequency of 5 Hz. Right: Fluorescent signals (ΔF/F) extracted from the 3 regions of interest (ROIs; black squares on images). In the ChR2 mouse heart APs were optogenetically induced while in CTRL mouse heart APs were spontaneously generated from the sinoatrial node (sinus rhythm). Crosstalk between ElectroFluor730p and blue light (due to VSD excitation at 470 nm light) was observed exclusively at the stimulation site (ROI 1). Scale bar: 2 mm. FIG. 5D: Left: optical mapping during four patterns of optogenetic stimulation: single-point at the apex, at the base, and in the middle of the heart and whole ventricle, indicated by the blue circles on the fluorescence image (F0) of the ChR2 mouse heart stained with the ElectroFluor730p. The mouse heart was optically paced using 3 ms blue light pulse at a stimulation frequency of 5 Hz (4.0 mW/mm2). Five representative frames of optical mapping (F/F0) showing the electrical activation in red and the baseline in green. Right: activation maps showing AP wave-front propagation. Scale bar: 2 mm.

### DETAILED DESCRIPTION

The disclosed hemicyanine dyes contain an amino-chromene moiety as an electron donor. The oxygen in the chromene imparts additional electron donor potency over simpler aromatic amines. Thus, the disclosed compounds are red shifted compared to corresponding aniline or naphthylamine donor moieties in hemicyanines. In addition to sensing rapid changes in membrane potential. the disclosed chromophores can be used as sensors of their molecular environments. For example, the compounds show almost no fluorescence in aqueous solution, moderate fluorescence in organic solvents such as ethanol and strong fluorescence in non-polar lipid environments. Absorbance and emission spectra of the compounds are shifted in these different environments. The compounds can be coupled to reactive groups, such as maleimide or succinimidyl ester, to allow for covalent labeling of proteins to enable detection of conformational changes or protein translocation as changes in fluorescence. The compounds are also be useful in non-biomedical applications such as in optoelectronics or counterfeit product detection, for example as an organic light emitting diode or as a detector for a target analyte.

### Definitions

“Alkyl” refers to a branched or unbranched saturated hydrocarbon group of 1 to 24 carbon atoms, such as methyl, ethyl, n-propyl, isopropyl, n-butyl, isobutyl, s-butyl, 1-butyl, n-pentyl, isopentyl, s-pentyl, neopentyl, hexyl. heptyl, octyl, nonyl, decyl, dodecyl, tetradecyl, hexadecyl, eicosyl, tetracosyl, and the like. The alkyl group can be cyclic or acyclic. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. “Alkyl” can be a C₁ alkyl, C₁-C₂ alkyl, C₁-C₃ alkyl, C₁-C₄ alkyl, C₁-C₅ alkyl, C₁-C₆ alkyl, C₁-C₇ alkyl, C₁-C₈ alkyl, C₁-C₉ alkyl, C₁-C₁₀ alkyl, C₁-C₁₂ alkyl, and the like up to and including a C₁-C₂₄ alkyl. “Heteroalkyl” refers to an alkyl group in which one or more of the hydrogen atoms bonded to carbon are substituted with a heteroatom including but not limited to O, S, or N(R)₂, in which each R can independently be hydrogen or a non-hydrogen substituent.

“Cycloalkyl” refers to a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. “Heterocycloalkyl” is a non-aromatic carbon-based ring type of cycloalkyl group, where at least one of the carbon atoms of the ring is replaced with a heteroatom such as, but not limited to, nitrogen, oxygen, sulfur, or phosphorus. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol.

“Bicyclic cycloalkyl” or “bicyclic heterocycloalkyl” refers to a compound in which two or more cycloalkyl or heterocycloalkyl groups are fused together. Non-limiting examples of bicyclic cycloalkyl groups include without limitation (1r,4r)-bicyclo[2.1.1]hexane, (1s,4s)-bicyclo[2.2.1]heptane, (1R,6S)-bicyclo[4.2.0]octane, adamantane, and the like. Non-limiting examples of bicyclic heterocycloalkyl groups include without limitation any of the foregoing groups in which at least one of the carbon atoms is replaced with a heteroatom such as nitrogen, oxygen, sulfur, or phosphorus.

“Alkenyl” refers to a hydrocarbon having from 2 to 24 carbons (e.g., 2-12 carbons) with a structural formula containing at least one carbon-carbon double bond. Asymmetric structures such as (A¹A²)C═C(A³A⁴) are intended to include both the E and Z isomers. The alkenyl group can be substituted with one or more groups including alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, among others.

“Cycloalkenyl” refers to a non-aromatic carbon-based ring composed of at least three carbon atoms and containing at least one carbon-carbon double bound, i.e., C═C. Examples of cycloalkenyl groups include cyclopropenyl, cyclobutenyl, cyclopentenyl, cyclopentadienyl, cyclohexenyl, cyclohexadienyl, norbornenyl, among others. The term “heterocycloalkenyl” is a type of cycloalkenyl group and is included within the meaning of the term “cycloalkenyl,” where at least one of the carbon atoms of the ring is replaced with a heteroatom such as nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, among others.

“Alkynyl” means a hydrocarbon group of 2 to 24 carbon atoms (e.g., 2-12 carbon atoms) with a structural formula containing at least one carbon-carbon triple bond. The alkynyl group can be unsubstituted or substituted with one or more groups including alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, among others.

“Cycloalkynyl” refers to a non-aromatic carbon-based ring composed of at least seven carbon atoms and containing at least one carbon-carbon triple bound. Examples of cycloalkynyl groups include cycloheptynyl, cyclooctynyl, cyclononynyl, and the like. The term “heterocycloalkynyl” is a type of cycloalkenyl group and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, among others.

“Aryl” refers to a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. In addition, the aryl group can be a single ring structure or comprise multiple ring structures that either are fused ring structures or attached via one or more bridging groups such as a carbon-carbon bond. For example, aryl can include biaryl in which two aryl groups are bound together via a fused ring structure, as in naphthalene, or are attached via one or more carbon-carbon bonds, as in biphenyl.

“Heteroaryl” refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further non-limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

“Halide” refers to F, Cl, Br, or I. “Haloalkyl,” “haloalkenyl,” and the like refer to compounds or groups that include at least one halide substituent at any position.

When the term “about” precedes a numerical value, the numerical value can vary within ±10% unless specified otherwise.

### I. Compounds

Some of the embodiments of the compounds have the following structure:

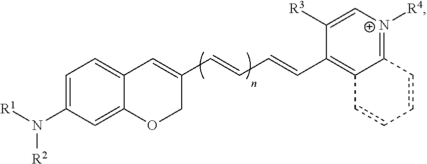

R¹-R³ are independently hydrogen, halide, C₁-C₁₂ alkyl, C₁-C₁₂ heteroalkyl, C₂-C₁₂ alkenyl, C₂-C₁₂ alkynyl, C₁-C₁₂ haloalkyl, C₂-C₁₂ haloalkenyl, cycloalkyl, heterocycloalkyl, bicyclic cycloalkyl, bicyclic heterocycloalkyl, cycloalkenyl, cycloalkynyl, aryl, or heteroaryl; wherein n is an integer ranging from 0-4. R⁴ is (C₁-C₁₂)—R⁵, where R⁵ is NR⁶⁻⁸, and R⁶-R⁸ are independently C₁-C₁₂ alkyl; sulfonate; carboxylic acid; amino; —C(O)O—R⁹, where R⁹ is succinimidyl; or —NH—C(O)—R¹⁰—R¹¹, where R¹⁰ is C₁-C₄ alkyl and R¹¹ is maleimide. The dashed line (- - - -) denotes an optional ring structure.

In one aspect, R¹ and R² are independently C₁-C₁₂ alkyl. In a further aspect, R¹ and R² are independently C₁-C₁₀ alkyl. In a further aspect, R¹ and R² are independently C₁-C₈ alkyl. In a further aspect, R¹ and R² are independently C₁-C₆ alkyl, e.g., C₃-C₆ alkyl. In a further aspect, R¹ and R² are independently C₁-C₄ alkyl.

In a further aspect, n is 0 or 1. In a further aspect, R³ is hydrogen or halide. In a still further aspect, R³ is hydrogen of fluoride.

It is understood that the positively charged nitrogen (attached to R⁴) will be opposed by a counterion, whether free or as part of R⁴. The counterion can be any suitable counterion, such as halide, carboxylate, sulfonate, and the like.

In some aspects, R⁴ has the structure:

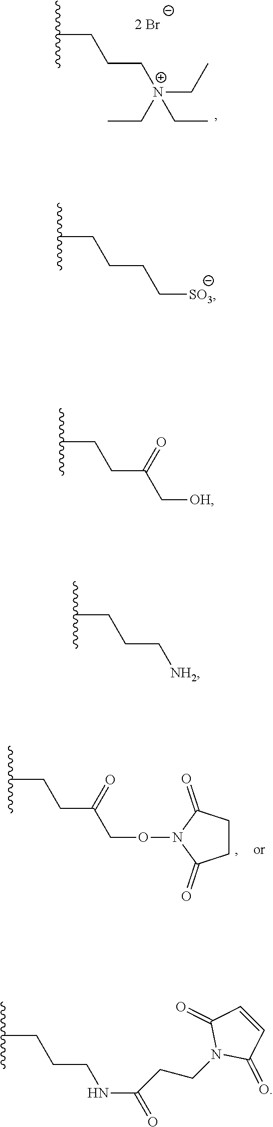

Specific embodiments of the disclosed compounds have the following structure:

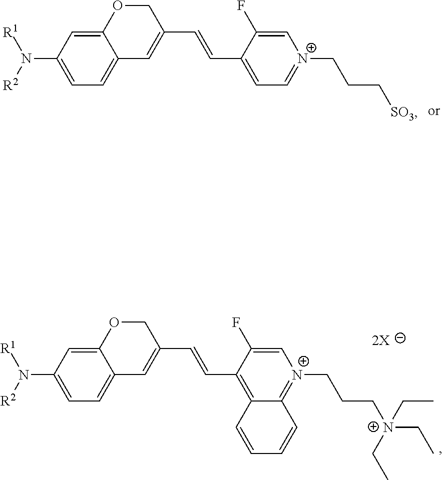

X is a halide. R¹ and R² are independently n-butyl, n-pentyl, or n-hexyl. In one aspect, each of R¹ and R² include the same alkyl group, which can n-butyl, n-pentyl, or n-hexyl.

### II. Methods of Making the Compounds

In one aspect, the chromene based aldehyde precursors to the compounds can be prepared according to Scheme 1. In some aspects, the last step can be repeated to extend the length of the conjugated chain (e.g., to obtain compounds in which n=1-4).

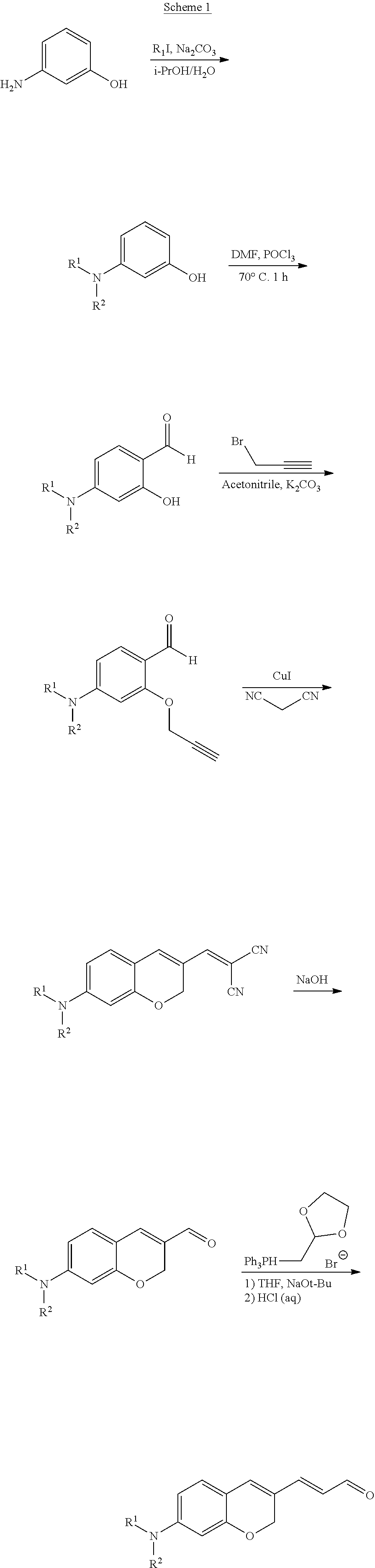

In a further aspect, the compounds can be prepared from the corresponding aldehyde precursors according to Scheme 2. When R³ contains a reactive group such as a carboxylic acid or an amine, the compounds can optionally be coupled to reactive such as maleimide or succinimidyl ester to allow for covalent labeling of proteins to detect conformational changes or protein translocation as changes in fluorescence. The condensation reaction can be carried out for example in a polar solvent such as ethanol, at elevated temperatures, e.g., 120-140° C., and in some embodiments in the presence of a pyrrolidone catalyst.

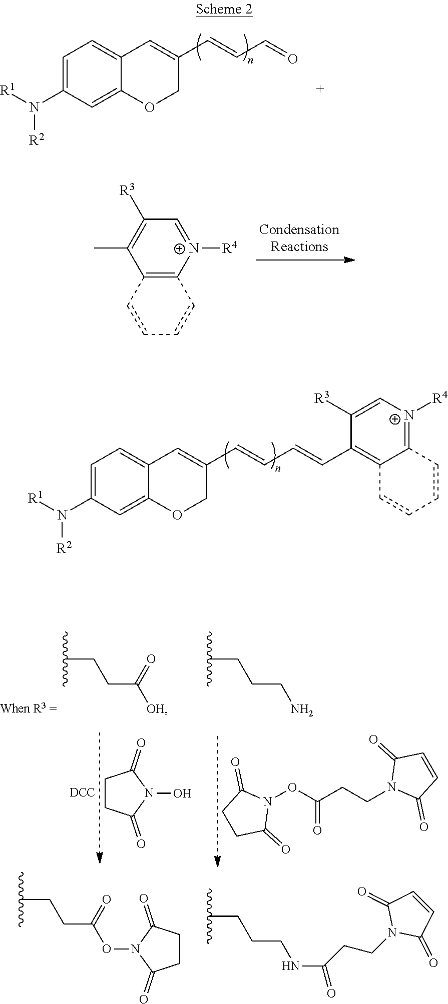

### III. Applications of Compounds

In some embodiments, the compounds have an optimal voltage sensitivity at an excitation wavelength of about 660 nm as measured on a voltage-clamped hemispherical bilayer. In other embodiments, the compounds have an optimal voltage sensitivity at an excitation wavelength of about 730 nm as measured on a voltage-clamped hemispherical bilayer.

Optimal voltage sensitivity excitation is defined as the excitation wavelength at which the change in fluorescence divided by the baseline fluorescence (ΔF/F) is at a maximum value. This value can be expressed as a % per 100 mV. In one embodiment, the compounds have an optimal voltage sensitivity value of about −15 to −18% per 100 mV. In another embodiment, the compounds have an optimal voltage sensitivity value of about 12% per 100 mV.

It is known that optimal voltage sensitivity can be obtained by measuring the compounds on a voltage-clamped hemispherical bilayer apparatus. The apparatus can include a patch-clamp amplifier, such as a HEKA EPC-8 patch-clamp amplifier. Measurements can be obtained by stepping the membrane potential between two different potentials for a set time lapse. such as, for example, −110 and −60 mV every 10 ms.

The compounds are useful in a variety of applications, including imaging applications. In one embodiment, disclosed are methods of recording ratiometric voltage with simultaneous optogenetic actuation using a disclosed compound. An advantage of the disclosed compounds compared to traditional voltage sensitive dyes are their long spectral windows, which reduces the amount of artifacts in such measurements due to overlapping spectral signals. Because of the long spectral windows, the disclosed compounds can be useful for techniques such as dual-wavelength ratiometry. Dual-wavelength ratiometry can be accomplished using excitation wavelength selection, excitation-based ratiometry, emission wavelength filtering, emission-based ratiometry, or shifted emission excitation ratioing, also known as “SEER” imaging.

The disclosed compounds can also be useful in applications that combine voltage sensitive dyes (VSDs) with other optical technologies, such as calcium or optogenetic activation.

Also disclosed are methods of imaging tissue with the use of a disclosed compound. Because the disclosed compounds are long wavelength VSDs, they are advantageous over traditional VSDs due to the avoidance of interference by hemoglobin in blood perfused and in vivo cardiac imaging. This advantage also makes the disclosed compounds useful for thick tissue imaging, due to the reduction of light scattering. In addition to cardiac tissue, other tissue types and/or organs may be imaged by one or more of the disclosed compounds.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

### EXAMPLES

The following examples further illustrate this disclosure. The scope of the disclosure and claims is not limited by the scope of the following examples.

Longer wavelength chromophores can be designed by extending the conjugated π-system. For the disclosed hemicyanines, this was achieved by using naphthylamine (instead of aniline) donor moieties and quinolinium acceptors (instead of pyridinium); incrementing the number of double bonds in the linker moieties produces additional red shifts. However, increasing the size of the π-system in this way can reduce the solubility of the VSDs and also decrease photostability. Previously, fluorine substituents have been employed to red shift the spectra without further increases in the size of the π-system; indeed, fluorination intrinsically also imparts improved photostability.

### I. Mouse Heart Imaging

All animal handling and procedures were performed in accordance with the guidelines from Directive 2010/63/EU of the European Parliament on the protection of animals used for scientific purposes. The experimental protocol was approved by the Italian Ministry of Health (protocol number 944/2018-PR). Transgenic mice (ChR2-mhc6-cre+) with cardiomyocyte-specific expression of Channelrhodopsin-2 (ChR2, H134R variant) were generated and employed in this study. Wild-type mice (C57BL/6J) and ChR2-mhc6-cre+ mice (6 months old) were heparinized (0.1 mL at 5000 units/mL) and anesthetized by inhaled isoflurane (5%). The excised heart was immediately bathed in Krebs-Henseleit (KH) solution and cannulated through the aorta. The KH buffer contained (in mM): 120 NaCl, 5 KCl, 2 MgS₂ O₄-7H₂O, 20 NaHCO₃, 1.2 NaH₂PO₄—H₂O, 1.8 CaCl₂ and 10 glucose, pH 7.4 when equilibrated with carbogen. Cardiac contraction was inhibited during the entire experiment with 1 μM blebbistatin (Enzo Life Sciences, Farmingdale, NY, USA) in the solution. The cannulated heart was perfused through the aorta (using a horizontal-Langendorff perfusion system) with the KH solution and then transferred to a custom-built optical mapping chamber at a constant flow of 2.5 mL/min at (36±0.5)° C. Two platinum electrodes were placed below the heart for monitoring cardiac electrical activity via electrocardiogram (ECG). 1 mL of perfusion solution containing the voltage sensitive dye (VSD; ElectroFluor730p: 10 μM; di-4-ANBDQPQ: 6 μg/mL) was bolus injected into the aorta. All the experiments were performed at (36±0.5° C.) within 1 hour after dye loading to avoid potential re-distribution of the dye and accumulation of phototoxic by-products.

### II. Cell Isolation and Patch Clamp Recording

Ventricular cardiomyocytes from ChR2 mice were isolated by enzymatic dissociation. The excised heart was immediately bathed in cell isolation buffer and the proximal aorta was cannulated for perfusion in a Langendorff system. Buffer solution contained (in mM): 120 NaCl, 1.2 MgCl₂, 10 KCl, 1.2 KH₂PO₄, 10 glucose, 10 HEPES, 20 taurine, 5 pyruvate, pH 7.4 (adjusted with NaOH), oxygenated with oxygen. After perfusion at 36° C. for 15 minutes with a constant flow of 3 mL/min, the solution was then switched to a recirculating enzyme solution, made from the same buffer with the addition of 0.1 mg/mL LIBERASE (Roche Applied Sciences, Penzberg, Germany). After 8 minutes, the ventricles were excised and cut into small pieces in buffer solution added with 1 mg/mL of bovine serum albumin (BSA). Gentle stirring was used to further facilitate dissociation of myocytes. The cell suspension was left to settle, and the cell pellet was resuspended in Tyrode buffer, containing (in mM): 133 NaCl, 4.8 KCl, 1.2 MgCl₂, 10 glucose and 10 HEPES, pH 7.4 (adjusted with NaOH). The calcium concentration of the cell suspension was gradually increased to 0.6 mM by adding 15 μL of CaCl₂ (0.1 M). Finally, cardiomyocytes were superfused with Tyrode buffer containing 1.8 mM CaCl₂ during patch-clamp experiments. Patch-clamp data recordings and analysis were performed using a Multiclamp700B amplifier in conjunction with pClamp 10.0 and a DigiData 1440A AD/DA interface (Molecular Devices, San Jose, CA, USA). For resting membrane potential (Vᵣₑₛₜ) and AP recordings, the pipette solution contained (in mM): 130 potassium aspartate, 0.1 Na-GTP, 5 Na₂-AT, 11 EGTA, 5 CaCl₂, 2 MgCl₂, 10 HEPES (pH 7.2 with KOH). Intracellular access was obtained via whole-cell ruptured patch. Action potentials (APs) were optogenetically-elicited with short (3 ms) blue light pulses at a stimulation frequency of 2 Hz using a light-emitting diodes (LED) operating at 470 nm (SPECTRA X light engine, Lumencor, Beaverton, OR, USA) at 6mW/mm² and a ×20 objective (NA; 0.5, HCX PL FLUOTAR, Leica Microsystems, Wetzlar, Germany). Cardiomyocytes were also continuously illuminated using amber light (590/15 nm) at 0.7 mW/mm². All experiments were performed at (36±0.5)° C.

### III. Optical Mapping

Optical mapping was performed using a custom-made mesoscope. Stained mouse hearts were illuminated in a wide-field configuration using a 2× objective (TL2x-SAP, Thorlabs, Newton, NJ, USA) and employing four different LED operating at a wavelength centred at 730 nm, 625 nm, 590 nm and 530 nm (M730L5, M625L3, M590L4 and M530L4 Thorlabs, Newton, NJ, USA) followed by excitation band-pass filters at 730/39 nm, 640/40 nm, 589/15 nm (FF01-730/39-25, FF01-640/40-25, FF01-589/15-25, Semrock) and 530/43 nm (MF530-43, Thorlabs) respectively. di-4-ANBDQPQ stained hearts were excited at 625 nm (0.875 mW/mm²) and a dichroic beam splitter (FF685-Di02-25×36, Semrock) followed by a band-pass filter at 775/140 nm (FF01-775/140-25, Semrock) was used for collecting the emitted fluorescent signal. ElectroFluor730p stained hearts were excited using three different wavelengths 730, 590 and 530 nm (at 1.07, 0.385 and 0.794 mW/mm² respectively) with a dichroic beam splitter (FF757-Di01-25×36, Semrock) and band-pass filter at 792/64 nm (FF01-792/64-25, Semrock). A ×20 objective (LD Plan-Neofluar ×20/0.4 M27, Carl Zeiss Microscopy, Oberkochen, Germany) was used to focus the fluorescent signal on a central portion (128×128 pixels) of the sensor of a sCMOS camera (OrcaFLASH 4.0, Hamamatsu Photonics, Shizuoka, Japan) operating at a frame rate of 1 kHz (1 ms actual exposure time). The detection path allows a field of view (at the object space) of 10.1×10.1 mm² sampled with a pixel size of 80 μm. Optogenetic pacing was performed employing a Texas Instruments Lightcrafter 4500 projector (Dallas, TX, USA) operating at 450/40 nm (3 ms pulse duration at 4 mW/mm²). Light intensities were measured at sample site using a photodiode sensor (PD300-3W, Ophir Optronics, Jerusalem, Israel). Hearts were electrically paced at the apex with a bipolar electrode using an isolated constant voltage stimulator (DS2A, Digitimer, Welwyn Garden City, Hertfordshire, UK).

### IV. Data and Image Analysis

All programs for data acquisition and analysis were developed with LabVIEW (National Instruments). For optical recordings, ΔF/F imaging of cardiac electrical activity was performed by processing raw data: for each frame, the mean baseline was subtracted, and the frame was subsequently normalized to the mean baseline, yielding a percentage change in fluorescence over time. For each heart, AP kinetics parameters were measured, trace by trace, in order to get the mean values after averaging 10 subsequent trials. AP amplitude (APA), AP maximum rising slope (APRS), AP duration (APD) at 50% of repolarization (APD50), APD at 70% of repolarization (APD70) were measured in a selected region of interest (ROI) of 10×10pixels (˜1 mm²). APD was determined relative to the time of maximum depolarization. The activation map was generated as follow: after a spatial binning of 6×6 pixels, a seed reference pixel was arbitrarily chosen, and the cross-correlation of the fluorescence trace was calculated pixel by pixel, in order to estimate the temporal shift among every pixel. Graphic representation of data was obtained using OriginPro 2018, version 9.5 64-bit (OriginLab Corporation, Northampton, MA USA).

### V. Synthesis

The general synthetic scheme is shown below.

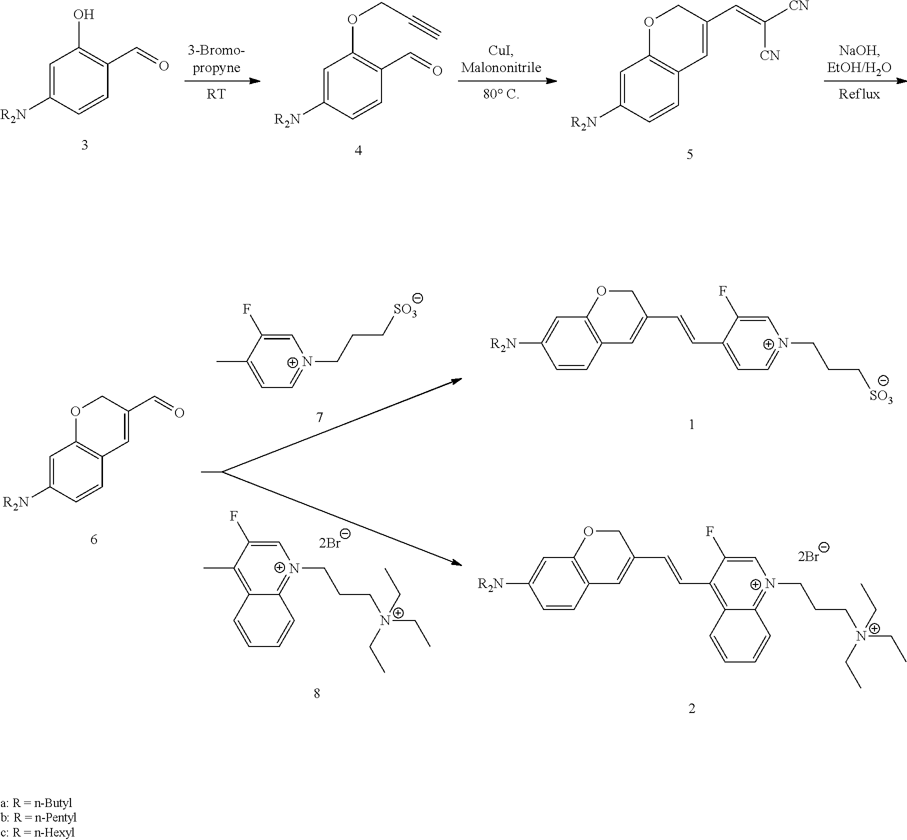

All chemicals were purchased from commercial sources (Sigma Aldrich, Acros, TCI America, Combi-Blocks, and others) and used without further purification. Column chromatography was generally performed on COMBIFLASH NextGen 300 using normal phase silica columns except that for some final dye products amine silica columns were used. Thin layer chromatography (TLC) was carried out on EM 60 F-254 plastic TLC plates. NMR spectra were recorded on Varian 400 MHz, 500 MHz, and 800 MHz instruments at the UConn Heath Structural Biology Facility. All spectra are referenced internally to TMS or residual solvent signals. Electrospray ionization (ESI) high-resolution mass spectra (HRMS) were obtained on a QStar Elite (AB Sciex) at the Department of Chemistry, University of Connecticut. UV/Vis absorption spectra were recorded on a UV-Visible spectrophotometer (Shimadzu, UV-1601PC) and fluorescence emission spectra on a Horiba Fluorolog spectrofluorometer (Horiba, Inc., Edison, NJ). Fluorescence quantum yield for dyes was determined by a relative method using Rhodamine 6G (excited at 480 nm, FQY=0.95 in EtOH) or cresyl violet (excited at 580 nm, FQY=0.54 in MeOH) as the references.

While 3a is commercially available from TCI America, 3b and 3c were synthesized from commercial products by the following two step procedure:

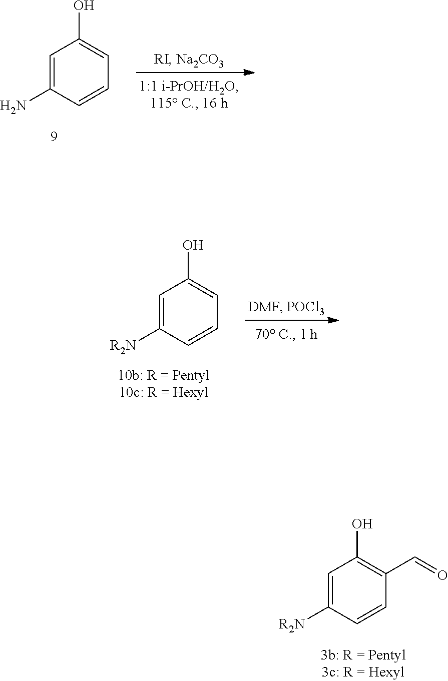

i) 9→10. A mixture of 3-aminophenol (30 mmol), 1-iodoalkane (63 mmol), Na₂CO₃ (42 mmol), i-PrOH (10 mL), and H₂O (10 mL) was sealed in a pressure vessel, and stirred at 115° C. overnight (16 h). Upon cooling down the top organic layer was separated, and the aqueous layer was extracted with more ethyl acetate (3×30 mL). The ethyl acetate extracts were combined, concentrated under vacuum, and then purified by column chromatography (SiO₂, CH₂Cl₂ to 1:9 EtOAc/CH₂Cl₂) to obtain the product.

3-(Dipentylamino) phenol (10b). Light gray oil, yield 4.2 g (56%). R_{f}(silica gel, 3:7 EtO Ac/Hexane)=0.59; ¹H NMR (400 MHz, CDCl₃): δ 7.04 (t, J=8.4 H7, 1H), 6.23 (d, J=7.6 Hz, 1H), 6.06-6.16 (m, 2H), 4.61 (br, 1H), 3.22 (t, J=7.6 Hz, 4H), 1.57 (m, 4H), 1.23-1.41 (m, 8 H), 0.91 (t, J=7.0 Hz, 6H); HRMS (ESI+): m/z=250.2143. [M+H]⁺ (calcd for C₁₆H₂₈NO⁺: 250.2165).

3-(Dihexylamino) phenol (10c). Light gray oil, yield 6.05 g (73%). R_{f}(silica gel, 3:7 EtOAc/Hexane)=0.64; ¹H NMR (400 MHz, CDCl₃): δ 7.04 (t, J=8.0 Hz, 1H), 6.23 (d, J=7.6 Hz, 1H), 6.06-6.18 (m, 2H), 4.67 (br, 1H), 3.21 (t, J=8.0 Hz, 4H), 1.56 (m, 4H), 1.26-1.40 (m, 12 H), 0.89 (t, J=6.6 Hz, 6H); HRMS (ESI+): m/z=278.2473 [M+H]⁺ (calcd for C₁₈H₃₂NO⁺: 278.2478).
ii) 10→3. To a 50 mL flask were added 7 mL anhydrous DMF, and then 3 mL POCl₃ (32 mmol) dropwise at room temperature under N_{2.} The mixture was cooled down to 0° C., and then 10 (8 mmol) was added as a solution in 3 mL DMF. The mixture was allowed to react at RT for 1 h, and then at 70° C. for 1 h. Upon cooling down 20 mL H₂O and 20 mL ethyl acetate were added, and K₂CO₃ was added to neutralize the aqueous solution until pH 7. The organic phase was separated and the aqueous phase was extracted with more ethyl acetate (20 mL×2). The ethyl acetate extracts were combined, dried with anhydrous Na₂SO₄, concentrated under vacuum, and then purified by column chromatography (SiO₂, CH₂Cl₂ to 1:9 EtOAc/CH₂Cl₂) to obtain the product.

4-(Dipentylamino)-2-hydroxy-benzaldehyde (3b). Light brown oil, yield 1.86 g (84%). R_{f}(silica gel, 3:7 EtOAc/Hexane)=0.65; ¹H NMR (400 MHz, CDCl₃): δ 11.64 (s, 1H), 9.48 (s, 1H), 7.25 (d, J=9.2 Hz, 1H), 6.22 (dd, J=2.4 Hz, 8.8 Hz, 1H), 6.04 (d, J=2.4 Hz, 1H), 3.31 (t, J=7.8 Hz, 4H), 1.61 (m, 4H), 1.28-1.42 (m, 8 H), 0.92 (t, J=7.2 Hz, 6H); HRMS (ESI+): m/z=278.2103. [M+H]⁺ (calcd for C₁₇H₂₈NO₂⁺: 278.2115).

4-(Dihexylamino)-2-hydroxy-benzaldehyde (3c). Reddish Oil, yield 1.94 g (79%). R_{f}(silica gel, 3:7 EtOAc/Hexane)=0.74; ¹H NMR (400 MHz, CDCl₃): δ 11.64 (s, 1H), 9.48 (s, 1H), 7.25 (d, J=9.2 Hz, 1H), 6.22 (dd, J=2.4 Hz, 8.8 Hz, 1H), 6.03 (d, J=2.4 Hz, 1H), 3.30 (t, J=8.0 Hz, 4H), 1.60 (m, 4H), 1.28-1.40 (m, 12 H), 0.90 (t, J=6.8 Hz, 6H); HRMS (ESI+): m/z=306.2422 [M+H]⁺ (calcd for C₁₉H₃₂NO₂⁺: 306.2428).

### 3-Fluoro-4-methyl-1-(3-sulfopropyl)-pyridinium Hydroxide, Inner Salt (7)

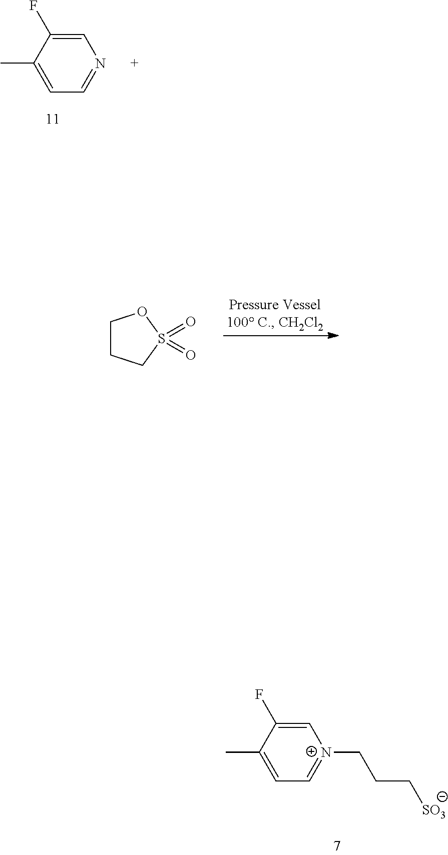

A mixture of 3-fluoro-4-methylpyridine (0.22 g, 2.0 mmol) and 1,3-propane sultone (0.244 g, 2.0 mmol) in 1 mL dichloromethane was sealed in a pressure vessel and stirred at 100° C. for 1 h. Upon cooling down the precipitates formed were filtered out, washed with CH₂Cl₂ to give 7 as a white power (0.28 g, 60%). ¹H NMR (500 MHz, CD₃OD): δ 9.15 (dd, J=1.3 Hz, 4.3 Hz, 1H), 8.79 (dd, J=1.0 Hz, 6.0 Hz, 1H), 8.05 (t, J=6.5 Hz, 1H), 4.79 (t, J=7.5 Hz, 2H), 2.86 (t, J=6.8 Hz, 2H), 2.62 (d, J=1.5 Hz, 3H), 2.44 (m, 2H); HRMS (ESI+): m/z=.[M+H]⁺ (calcd for C₉H₁₃FNO₃S⁺: 234.0595).

### 3-Fluoro-4-methyl-1-[3-(triethylammonio)propyl]-quinolinium Dibromide (8)

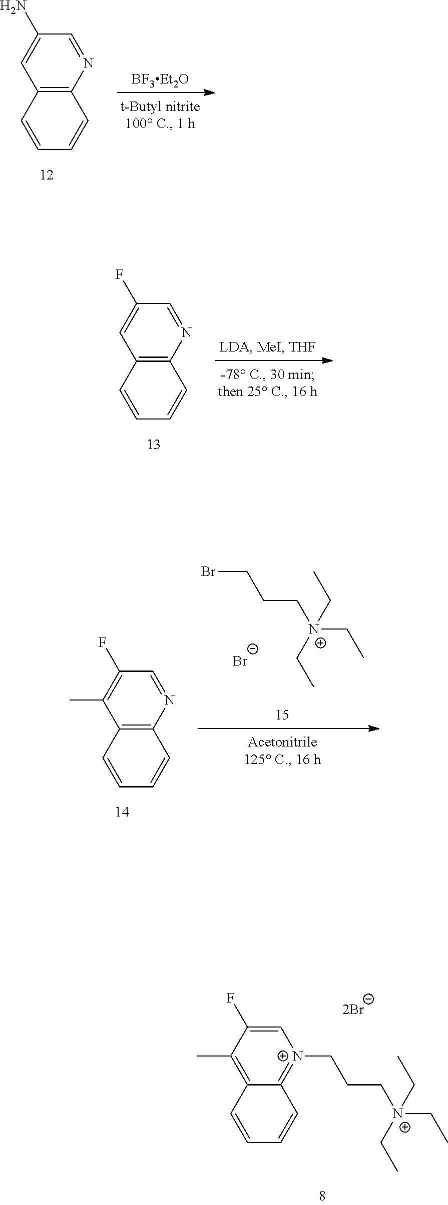 [SMILES: Nc1cnc2ccccc2c1 ; Fc1cnc2ccccc2c1 ; Cc1c(F)cnc2ccccc12 ; CC[N+](CC)(CC)CCCBr ; [Br-] ; CC[N+](CC)(CC)CCC[n+]1cc(F)c(C)c2ccccc21]

i) 12→13. Borontrifluoride etherate (7 mL, 55 mmol) was added to a suspension of 3-aminoquinoline (12, 4.68 g, 32 mmol) in 40 mL 1,2-dichlorobenzene at room temperature. The mixture was heated to 100° C. and then t-butylnitrite (7 mL, 58 mmol) was added dropwise. After 1 h the mixture was allowed to cool down, and the solvent was decanted out, leaving red viscous oil behind. Dichloromethane (25 mL×2) was added and then decanted out to remove dichlorobenzene cleanly. Water (20 mL) was added to dissolve the mixture, K₂CO₃ was added to basify the aqueous solution, and then aqueous phase was extracted with ethyl acetate (50 mL×3). The organic phases were combined, dried with anhydrous MgSO_{4,} concentrated under vacuum, and then purified by column chromatography (SiO₂, CH₂Cl₂). A final purification by vacuum distillation gave 3-fluoroquinoline (13) as a colorless oil (2.76 g, 58%). R_{f}(silica gel, 1:1 EtOAc/Hexane)=0.69; ¹H NMR (400 MHz, CDCl₃): δ 8.83 (d, J=2.8 Hz, 1H), 8.13 (d, J=8.4 Hz, 1H), 7.76-7.83 (m, 2H), 7.69 (t, J=8.0 Hz, 1H), 7.59 (t, J=8.0 Hz, 1H); HRMS (ESI+): m/z=148.0537 [M+H]⁺ (calcd for C₉H₇FN⁺: 148.0557).
ii) 13→14. To a stirred solution of diisopropylamine (1.13 mL, 8.3 mmol) in 15 mL anhydrous tetrahydrofuran at −78° C. was added dropwise a 2.5 M n-BuLi solution in hexane (2.8 mL, 7.0 mmol). The mixture was stirred at this temperature for 30 min more and then 3-fluoroquinoline (13, 0.735 g. 5 mmol) was added dropwise. The solution turned deep yellow and then yellow precipitates formed. After 30 min Mel (0.34 mL, 5.5 mmol) was added dropwise, and the mixture was allowed to warm up to room temperature overnight. 30 mL saturated NaHCO₃ solution was added and the aqueous phase was extracted with ethyl acetate (50 mL×3). The organic phases were combined, dried with anhydrous MgSO₄, concentrated under vacuum, and then purified by vacuum distillation to give 3-fluoro-4-methyl-quinoline (14) as a colorless oil that eventually turned into white needle crystals upon cooling down (524 mg, 65%). ¹H NMR (800 MHz, CDCl₃): δ 8.75 (s, 1H), 8.11 (d, J=8.0 Hz, 1H), 7.97 (d, J=8.0 Hz, 1H), 7.68 (t, J=8.0 Hz, 1H), 7.61 (t, J=7.2 Hz, 1H), 2.62 (d, J=2.4 Hz, 3H); HRMS (ESI+): m/z=162.0694 [M+H]⁺ (calcd for C₁₀H₉FN⁺: 162.0719).
iii) 14→8. To a 35 mL pressure vessel were added 3-fluoro-4-methyl-quinoline (14, 242 mg, 1.5 mmol), 3-bromopropyl-triethylammonium bromide (15, 455 mg, 1.5 mmol), and 5 mL acetonitrile. The mixture was heated at 125° C. and the acetonitrile was allowed to escape until only about 0.5 mL was left. The mixture was then sealed and allowed to react overnight. Upon cooling down the precipitates formed were filtered out and washed with diethyl ether to 8 as a white solid (382 mg, 55%). R_{f}(silica gel, 24:4:16:6:6 CH₂Cl₂/i-PrOH/MeOH/H₂O/HOAc)=0.10; ¹H NMR (400 MHz, CD₃OD): δ 9.84 (d, J=4.8 Hz, 1H), 8.72 (d, J=9.2 Hz, 1H), 8.63 (d, J=8.4 Hz, 1H), 8.30 (t, J=7.8 Hz, 1H), 8.12 (t, J=7.8 Hz, 1H), 5.25 (t, J=7.8 Hz, 2H), 3.64 (t, J=8.8 Hz, 2H), 3.41 (q, J=7.2 Hz, 6H), 2.99 (d, J=2.0 Hz, 3H), 2.59 (m, 2H), 1.36 (t, J=7.0 Hz, 6H); HRMS (ESI+): m/z=152.1145 [M−2Br]²⁺ (calcd for C₁₉H₂₉FN₂²⁺: 152.1152).

### 4-(Dialkylamino)-2-prop-2-ynoxy-benzaldehyde (4)

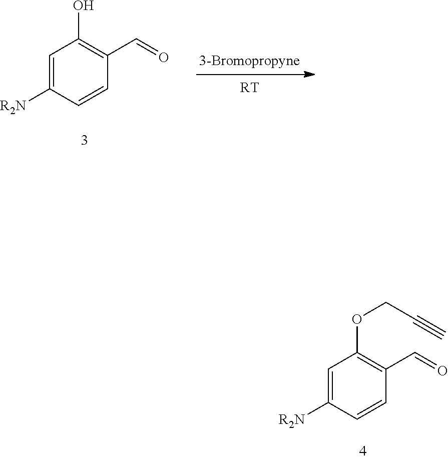

To a 50 mL flask were added 4-(dialkylamino)-2-hydroxy-benzaldehyde (3, 6.5 mmol), K₂CO₃ (1.8 g, 13 mmol), acetonitrile (20 mL), and 3-bromopropyne (1.16 g, 9.8 mmol) subsequently. The mixture was stirred at room temperature for 16 h. 20 mL water was added and the aqueous phase was extracted with dichloromethane (50 mL×2). The organic phases were combined, dried with anhydrous Na₂SO₄, concentrated under vacuum to give product 4 as light brown oil. This product was used for the next step reaction without further purification.

4a, R=n-butyl. Light brown oil, yield 1.83 g (98%). R_{f}(silica gel, EtOAc)=0.86; ¹H NMR (400 MHz, CDCl₃): δ 10.12 (s, 1H), 7.71 (d, J=8.8 Hz, 1H), 6.30 (d, J=8.8 Hz, 1H), 6.21 (s, 1H), 4.79 (s, 2H), 3.34 (t, J=7.8 Hz, 4H), 2.56 (s, 1H), 1.62 (m, 4H), 1.32-1.44 (m, 4 H), 0.98 (t, J=7.4 Hz, 6H).

4b, R=n-pentyl. Light brown oil, yield 2.0 g (98%). R_{f}(silica gel, 3:7 EtOAc/Hexane)=0.56; ¹H NMR (400 MHz, CDCl₃): δ 10.12 (s, 1H), 7.71 (d, J=8.8 Hz, 1H), 6.30 (dd, J=2.2 Hz, 9.2 Hz, 1H), 6.20 (d, J=2.2 Hz, 1H), 4.78 (d, J=2.4 Hz, 2H), 3.33 (t, J=8.0 Hz, 4H), 2.56 (t, J=2.4 Hz, 1H), 1.64 (m, 4H), 1.28-1.43 (m, 8 H), 0.93 (t, J=7.0 Hz, 6H); HRMS (ESI+): m/z=316.2271. [M+H]⁺ (calcd for C₂₀H₃₀NO₂⁺: 316.2271).

4c, R=n-hexyl. Purification by column chromatography (SiO₂, CH₂Cl₂ to 1:9 EtOAc/CH₂Cl₂) gave 4c as a light yellow oil (1.34 g, 60%). R_{f}(silica gel, 3:7 EtOAc/Hexane)=0.56; ¹H NMR (400 MHz, CDCl₃): δ 10.12 (s, 1H), 7.71 (d, J=8.8 Hz, 1H), 6.29 (dd, J=2.2 H7, 9.0 H7, 1H), 6.20 (d, J=2.2 H7, 1H), 4.78 (d, J=2.4 H7, 2H), 3.33 (t, J=7.8 H7, 4H), 2.55 (t, J=2.4 Hz, 1H), 1.64 (m, 4H), 1.29-1.40 (m, 12 H), 0.91 (t, J=6.8 Hz, 6H); HRMS (ESI+): m/z=344.2593 [M+H]⁺ (calcd for C₂₂H₃₄NO₂⁺: 344.2584).

### 2-[(7-Dialkylamino-2H-chromen-3-yl) methylene]propanedinitrile (5)

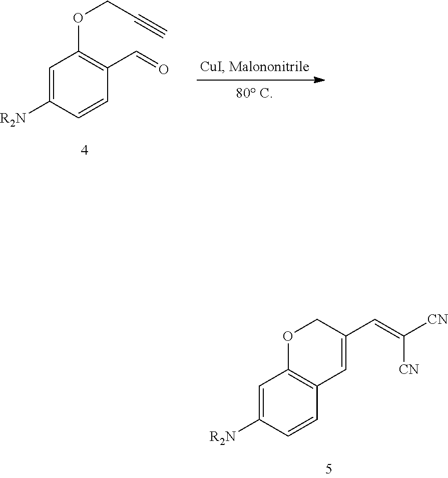

To a 50 mL Schlenk flask were added 4-(dialkylamino)-2-prop-2-ynoxy-benzaldehyde (4, 3.0 mmol), Cul catalyst (0.5 eq, 1.5 mmol), and 10 mL acetonitrile under N₂. The mixture was heated to 80° C. and then malononitrile (1.2 eq, 3.6 mmol) was added. The mixture was allowed to react for 18 h and it turned dark red. The mixture was then filtered through a fritted glass funnel to remove CuI catalyst, and the filtrate was concentrated and purified by column chromatography (SiO₂, CH₂Cl₂).

5a, R=n-butyl. Red oil, yield 332 mg (33%). R_{f}(silica gel, CH₂Cl₂)=0.35; ¹H NMR (400 MHz, CDCl₃): δ 7.08 (s, 1H), 7.02 (s, 1H), 6.97 (d, J=8.8 Hz, 1H), 6.26 (dd, J=2.4 Hz, 8.8 Hz, 1H), 6.07 (d, J=2.4 Hz, 1H), 5.33 (s, 2H), 3.32 (t, J=7.8 Hz, 4H), 1.59 (m, 4H), 1.32-1.42 (m, 4 H), 0.97 (t, J=7.4 Hz, 6H); HRMS (ESI+): m/z=336.2073. [M+H]⁺ (calcd for C₂₁H₂₆N₃O⁺: 336.2070).

5b, R=n-pentyl. Red oil, yield 327 mg (30%). R_{f}(silica gel, CH₂Cl₂)=0.50; ¹H NMR (400 MHz, CDCl₃): δ 7.07 (s, 1H), 7.01 (s, 1H), 6.97 (d, J=8.8 Hz, 1H), 6.25 (dd, J=2.4 Hz, 8.8 H7, 1H), 6.06 (d, J=2.4 Hz, 1H), 5.32 (s, 2H), 3.31 (t, J=7.8 H7, 4H), 1.61 (m, 4H), 1.26-1.42 (m, 8 H), 0.92 (t, J=7.0 Hz, 6H).

5c, R=n-hexyl. Red oil, yield 352 mg (30%). R_{f}(silica gel, CH₂Cl₂)=0.65; ¹H NMR (400 MHz, CDCl₃): δ 7.08 (s, 1H), 7.01 (s, 1H), 6.97 (d, J=8.8 Hz, 1H), 6.25 (dd, J=2.4 Hz, 8.8 Hz, 1H), 6.06 (d, J=2.4 Hz, 1H), 5.33 (s, 2H), 3.30 (t, J=7.8 Hz, 4H), 1.60 (m, 4H), 1.28-1.38 (m, 12 H), 0.90 (t, J=6.2 Hz, 6H); HRMS (ESI+): m/z=392.2674 [M+H]⁺ (calcd for C₂₅H₃₄N₃O⁺: 392.2696).

### 7-Dialkylamino-2H-chromene-3-carbaldehyde (6)

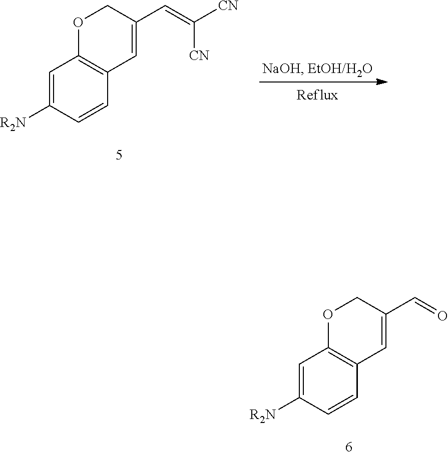

Compound 5 (0.9 mmol) was dissolved in 20 mL ethanol, and then NaOH (360 mg, 10 eq) in 20 mL H₂O was added. The mixture was refluxed for 1 h. Upon cooling down the ethanol was removed under vacuum and the aqueous phase was extracted with CH₂Cl₂ (50 mL×2). The organic phases were combined, dried with anhydrous MgSO₄, concentrated under vacuum, and then purified by column chromatography (SiO₂, CH₂Cl₂) to give compound 6.

6a. R=n-butyl. Yellow oil, yield 124 mg (48%). R_{f}(silica gel, CH₂Cl₂)=0.16; ¹H NMR (400 MHz, CDCl₃): δ 9.41 (s, 1H), 7.17 (s, 1H), 7.02 (d, J=8.8 Hz, 1H), 6.24 (dd, J=2.8 Hz, 8.8 Hz, 1H), 6.09 (d, J=2.4 Hz, 1H), 5.00 (s, 2H), 3.28 (t, J=7.8 Hz, 4H), 1.58 (m, 4H), 1.30-1.41 (m, 4 H), 0.96 (t, J=7.2 Hz, 6H); HRMS (ESI+): m/z=288.1965. [M+H]⁺ (calcd for C₁₈H₂₆NO₂⁺: 288.1958).

6b, R=n-pentyl. Yellow oil, yield 122 mg (43%). R_{f}(silica gel, CH₂Cl₂)=0.16; ¹H NMR (400 MHz, CDCl₃): δ 9.42 (s, 1H), 7.17 (s, 1H), 7.02 (d, J=8.8 Hz, 1H), 6.24 (dd, J=2.6 Hz, 8.6 Hz, 1H), 6.09 (d, J=2.4 Hz, 1H), 5.00 (s, 2H), 3.27 (t, J=7.8 Hz, 4H), 1.59 (m, 4H), 1.25-1.40 (m, 8H), 0.92 (t, J=7.2 Hz, 6H); HRMS (ESI+): m/z=316.2271. [M+H]⁺ (calcd for C₂₀H₃₀NO₂⁺: 316.2271).

6c, R=n-hexyl. Yellow oil, yield 235 mg (76%). R_{f}(silica gel, CH₂Cl₂)=0.35; ¹H NMR (400 MHz, CDCl₃): δ 9.41 (s, 1H), 7.17 (s, 1H), 7.02 (d, J=8.8 Hz, 1H), 6.24 (dd, J=2.8 Hz, 8.6 Hz, 1H), 6.09 (d, J=2.4 Hz, 1H), 5.00 (s, 2H), 3.27 (t, J=7.8 Hz, 4H), 1.58 (m, 4H), 1.28-1.36 (m, 12H), 0.90 (t, J=6.6 Hz, 6H); HRMS (ESI+): m/z=344.2593 [M+H]⁺ (calcd for C₂₂H₃₄NO₂⁺: 344.2584).

### Chromene-Based VSD 1

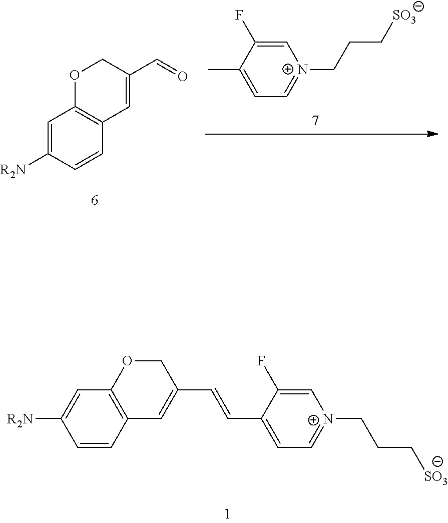

General procedure: To a pressure vessel were added aldehyde 6 (0.06 mmol), pyridinium 7 (0.06 mmol), pyrrolidine catalyst (1 μL), and ethanol (1 mL). The mixture were heated at 130° C. for 16 h and the solution turned purple. Upon cooling down the solvents were removed under vacuum and the residue was purified by column chromatography (SiO₂, CH₂Cl₂ to 15:85 MeOH/CH₂Cl₂) to give VSD 1.

1a, R=n-butyl. Purple solid, yield 10.5 mg (35%). R_{f}(silica gel, 24:4:16:6:6 CH₂Cl₂/i-PrOH/MeOH/HOAc/H₂O)=0.52; ¹H NMR (400 MHz, CD₃OD): δ 8.90 (d, J=4.8 Hz, 1H), 8.54 (d, J=7.2 Hz, 1H), 8.17 (t, J=7.2 Hz, 1H), 7.71 (d, J=16 Hz, 1H), 7.00 (m, 2H), 6.45 (d, J=16 Hz, 1H), 6.31 (d, J=8.0 Hz, 1H), 6.12 (s, 1H), 5.07 (s, 2H), 4.64 (t, J=7.2 Hz, 2H), 3.34 (t, J=6.0 Hz, 4H), 2.86 (t, J=6.8 Hz, 2H), 2.41 (m, 2H), 1.59 (m, 4H), 1.28-1.44 (m, 4H), 0.98 (t, J=7.4 Hz, 6H); HRMS (ESI+): m/z=503.2393 [M+H]⁺ (calcd for C₂₇H₃₆FN₂O₄S⁺: 503.2374).

1b, R=n-pentyl. Purple solid, yield 15.3 mg (48%). R_{f}(silica gel, 24:4:16:6:6 CH₂Cl₂/i-PrOH/MeOH/HOAc/H₂O)=0.76; ¹H NMR (400 MHz, CD₃OD): δ 8.91 (dd, J=1.4 Hz, 5.4 Hz. 1H), 8.54 (dd, J=1.2 Hz, 6.8 Hz, 1H), 8.17 (t, J=6.8 Hz, 1H). 7.72 (d, J=16 Hz, 1H), 7.02 (s, 1H), 7.01 (d, J=8.4 Hz, 1H), 6.46 (d, J=16 Hz, 1H), 6.31 (dd, J=2.4 Hz, 8.8 Hz, 1H), 6.12 (d, J=2.0 Hz, 1H), 5.08 (s, 2H), 4.65 (t, J=7.2 Hz, 2H), 3.34 (t, J=8.0 Hz, 4H), 2.86 (t, J=6.8 Hz, 2H), 2.41 (m, 2H), 1.61 (m, 4H). 1.28-1.44 (m, 8H), 0.94 (t, J=7.0 Hz, 6H); HRMS (ESI+): m/z=531.2657 [M+H]⁺ (calcd for C₂₉H₄₀FN₂O₄S⁺: 531.2687).

1c, R=n-hexyl. Purple solid, yield 7.7 mg (23%). R_{f}(silica gel, 24:4:16:6:6 CH₂Cl₂/i-PrOH/MeOH/HOAc/H₂O)=0.76; ¹H NMR (400 MHz, CD₃OD): δ 8.91 (dd, J=1.2 Hz, 5.6 Hz, 1H), 8.54 (dd, J=1.0 Hz, 6.6 Hz, 1H), 8.17 (t, J=7.0 Hz, 1H), 7.72 (d, J=16 Hz, 1H), 7.02 (s, 1H), 7.01 (d, J=8.8 Hz, 1H), 6.45 (d, J=16 Hz, 1H), 6.31 (dd, J=2.4 Hz, 8.8 Hz, 1H), 6.11 (d, J=2.4 Hz, 1H), 5.08 (s, 2H), 4.65 (t, J=7.2 Hz, 2H), 3.34 (t, J=7.2 Hz, 4H), 2.86 (t, J=6.8 Hz, 2H), 2.41 (m, 2H), 1.60 (m, 4H), 1.31-1.40 (m, 12H), 0.93 (t, J=7.0 Hz, 6H); HRMS (ESI+): m/z=559.2971 [M+H]⁺ (calcd for C₃₁H₄₄FN₂O₄S⁺: 559.3000).

### Chromene-Based VSD 2

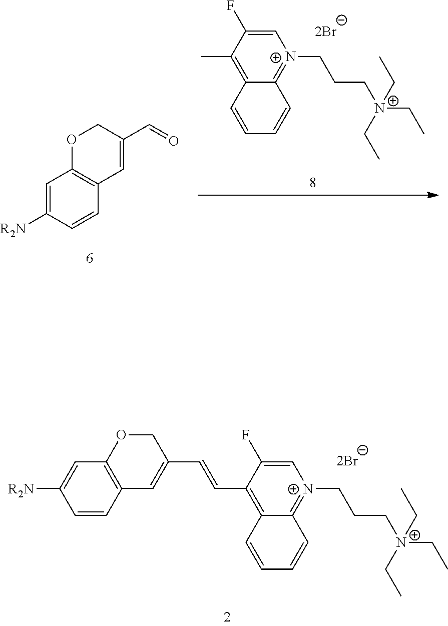

General procedure: To a Schlenk flask were added aldehyde 6 (0.02 mmol), quinolinium 8 (0.02 mmol), and n-butanol (0.5 mL) under N₂. The mixture was heated at 100° C. for 16 h. Upon cooling down the solvents were removed under vacuum and the residue was purified by column chromatography (SiO₂-amino, CH₂Cl₂ to 8:92 MeOH/CH₂Cl₂) to give VSD 2.

2a, R=n-butyl. Blue solid, yield 6.3 mg (43%). R_{f}(silica gel, 24:4:16:6:6 CH₂Cl₂/i-PrOH/MeOH/HOAc/H₂O)=0.51; ¹H NMR (400 MHz, CD₃OD): δ 9.35 (d, J=8.0 Hz, 1H), 8.81 (d, J=8.0 Hz, 1H), 8.43 (d, J=8.8 Hz, 1H), 8.18 (t, J=7.8 Hz, 1H), 7.98 (t, J=8.0 Hz, 1H), 7.98 (d, J=15.6 Hz, 1H), 7.14 (s, 1H), 7.07 (d, J=8.4 Hz, 1H), 7.05 (d, J=15.6 Hz, 1H), 6.36 (dd, J=2.6 Hz, 8.6 Hz, 1H), 6.16 (d, J=2.4 Hz, 1H), 5.28 (s, 2H), 4.98 (t, J=7.8 Hz, 2H), 3.53 (m, 2H). 3.34-3.42 (m, 10H), 2.48 (m, 2H), 1.61 (m, 4H), 1.28-1.45 (m, 13H), 0.99 (t, J=7.4 Hz, 6H); HRMS (ESI+): m/z=286.7031 [M−2Br]²⁺ (calcd for C₃₇H₅₂FN₃O₂⁺: 286.7042).

2b, R=n-pentyl. Blue solid, yield 10.8 mg (71%). R_{f}(silica gel, 24:4:16:6:6 CH₂Cl₂/i-PrOH/MeOH/HOAc/H₂O)=0.51; ¹H NMR (400 MHz, CD₃OD): δ 9.35 (d, J=7.6 Hz, 1H), 8.81 (d, J=8.8 Hz, 1H), 8.44 (d, J=8.4 Hz, 1H), 8.18 (t, J=7.6 Hz, 1H), 7.98 (t, J=8.0 Hz, 1H), 7.98 (d, J=15.6 Hz, 1H), 7.14 (s, 1H), 7.07 (d, J=8.4 Hz, 1H), 7.05 (d, J=15.6 Hz, 1H), 6.36 (dd, J=2.4 Hz, 8.6 Hz, 1H), 6.15 (d, J=2.4 Hz, 1H), 5.28 (s, 2H), 4.99 (t, J=8.0 Hz, 2H), 3.54 (m, 2H), 3.34-3.42 (m, 10H), 2.49 (m, 2H), 1.63 (m, 4H), 1.28-1.40 (m, 17H), 0.95 (t, J=7.0 Hz, 6H); HRMS (ESI+): m/z=300.7178 [M−2Br]²⁺ (calcd for C₃₉H₅₆FN₃O₂⁺: 300.7198).

2c, R=n-hexyl. Blue solid, yield 10.4 mg (66%). R_{f}(silica gel, 24:4:16:6:6 CH₂Cl₂/i-PrOH/MeOH/HOAc/H₂O)=0.51; ¹H NMR (400 MHz, CD₃OD): δ 9.37 (d, J=7.6 Hz, 1H), 8.79 (d, J=8.8 Hz, 1H), 8.44 (d, J=8.4 Hz, 1H), 8.17 (t, J=8.0 Hz, 1H), 7.97 (t, J=7.6 Hz, 1H), 7.96 (d, J=16 Hz, 1H), 7.12 (s, 1H), 7.06 (d, J=8.8 Hz, 1H), 7.02 (d, J=16 Hz, 1H), 6.35 (dd, J=2.2 Hz, 8.6 Hz, 1H), 6.14 (d, J=2.0 Hz, 1H), 5.27 (s, 2H), 5.00 (t, J=8.0 Hz, 2H), 3.55 (m, 2H), 3.34-3.42 (m, 10H), 2.49 (m, 2H), 1.62 (m, 4H), 1.28-1.42 (m, 21H), 0.93 (t, J=6.8 Hz, 6H); HRMS (ESI+): m/z=314.7325 [M−2Br]²⁺ (calcd for C₄₁H₆₀FN₃O²⁻; 314.7355).

### VI. Absorption and Fluorescence Properties

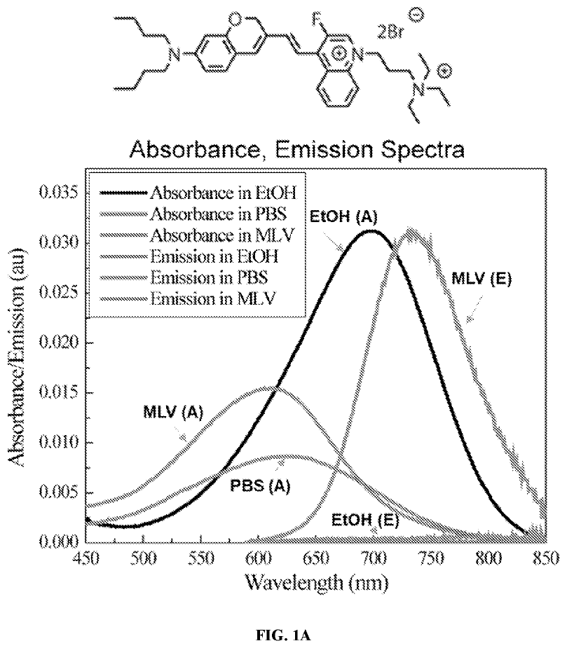

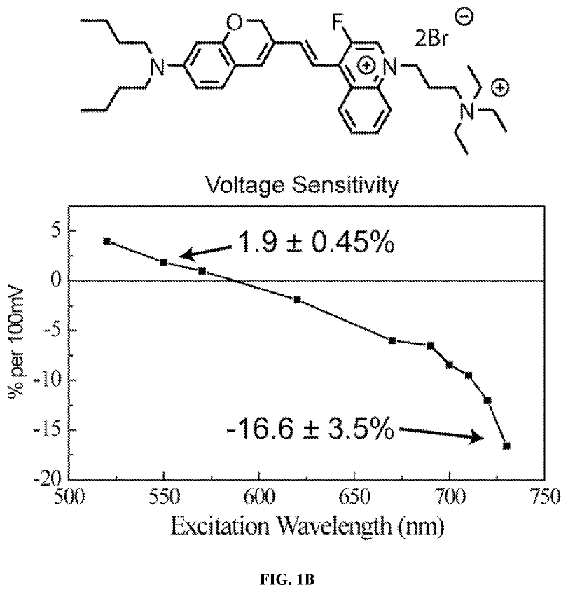

Characterization experiments were performed on the voltage-clamped hemispherical bilayer and the results are shown in FIG. 1A-1B. The disclosed chromene-based VSDs display an optimal voltage sensitivity that is red shifted about 100 nm relative to naphthylamine analogs. The optimal voltage sensitivity on the voltage clamped hemispherical bilayer is at 730 nm with a ΔF/F of −17%/100 mV; shows an inverted voltage sensitivity at 550 nm of +2%/100 mV, permitting ratiometric voltage imaging with optogenetic activation. The spectra show that this dye has good fluorescence when bound to membranes (MLV), but almost no fluorescence in ethanol (EtOH) or aqueous solution (PBS). This is a convenient feature of the disclosed hemicyanine VSDs, in that it reduces background fluorescence. The absorbance spectra display strong solvatochromism and the fluorescence has a large Stokes shift, also advantageous for fluorescence imaging.

Red-shifted VSDs will be of additional value to enable detection of fluorescence from deeper within the tissue; this is because light scattering, which limits tissue penetration. is proportional to inverse 4th power of the wavelength.

FIG. 1 provides the characteristics of the longer wavelength dye, demonstrating excellent voltage sensitivity at 730 nm, 100 nm higher than any previous VSDs. Similar results were obtained for compound la, albeit shifted to the red ˜30 nm. Without wishing to be bound by theory, it is believed that the disclosed compounds are the longest excitation wavelength voltage indicators (VSD or GEVI) reported that are able to resolve action potentials (some slow cyanine dyes that operate via a Nernstian redistribution mechanism have longer wavelengths). This will allow; for the first time, dual-wavelength ratiometric recording of voltage in conjunction with activation of CheRiff, which has optimal excitation at 460 nm but has a long red tail out to 550 nm. Long wavelength excitation will also have the advantage of permitting deeper light penetration into thick preparations, because of the inverse 4th power dependence of light scattering on wavelength, and the absence of endogenous chromophores with absorbance spectra in that region.

The disclosed chromophores extend the VSD measurements ˜100 nm further to the red than had been previously possible, with a demonstrated optimal sensitivity at 730 nm excitation. Because hemicyanine dyes are sensitive to their environment and can be used as polarity or membrane domain probes the disclosed chromophores will have applications beyond voltage sensing.

An advantage of the disclosed long-wavelength VSDs is that they are suitable for dual-wavelength ratio measurements and compatible with optogenetic actuators; ideal optogenetic actuators are excited with blue light but have spectra that tail to about 550 nm. The longest wavelength dye has optimal dual excitation at 470 nm and 630 nm (FIG. 1), so the lower wavelength would produce some unwanted activation of the light-sensitive channels. The disclosed chromene based chromophores will overcome this problem.

Three different alkyl groups, i.e. n-butyl, n-pentyl, and n-hexyl, have been incorporated as lipid membrane anchors to find a good balance between dye solubility and delivery in aqueous medium vs. persistent membrane staining for cells and biological tissues; which VSD to choose depends on the specific needs of the biological preparation. In general, the amino-chromene dyes are more water soluble and therefore less persistent on cells and tissues than their isostructural amino-naphthyl analogs; this issue was readily addressed by employing the dipentyl or dihexyl VSDs.

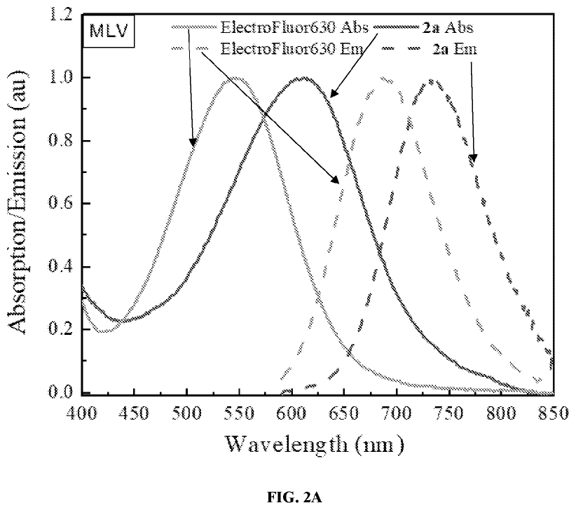

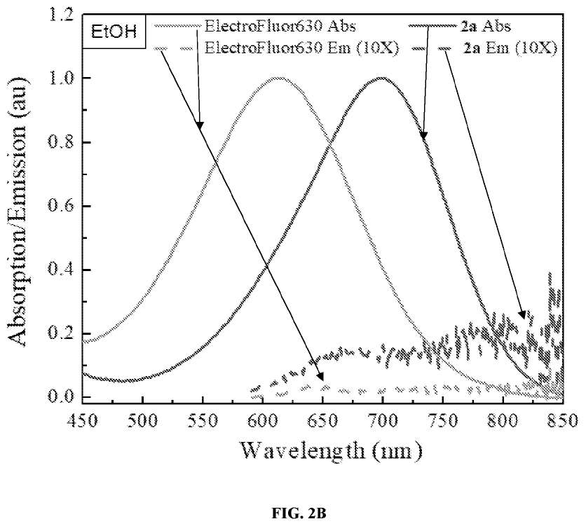

Absorbance and the fluorescence properties of the new VSDs were determined. The absorption and emission spectra for 2a, together with ElectroFluor630, in aqueous buffer (PBS), ethanol, and when intercalated in the membrane of lipid vesicles are shown in FIGS. 2A-C. Compound 2a displays a dramatic fluorescence enhancement of over 100-fold upon binding to lipid membranes. This is particularly advantageous for fluorescence imaging because background fluorescence from unbound dye is negligible. Another characteristic of the hemicyanine VSDs that is preserved by 2a in FIGS. 2A-C is the large Stokes shift between the excitation and emission maxima. 125 nm for the membrane-bound dye. This is also a benefit for fluorescence detection and imaging, as the fluorescence can be readily filtered from scattered exciting light. The broad wavelength window afforded by the spectra is also an important benefit for the design of experiments with multiple fluorescent sensors. For comparative purposes, certain VSDs are shown below.

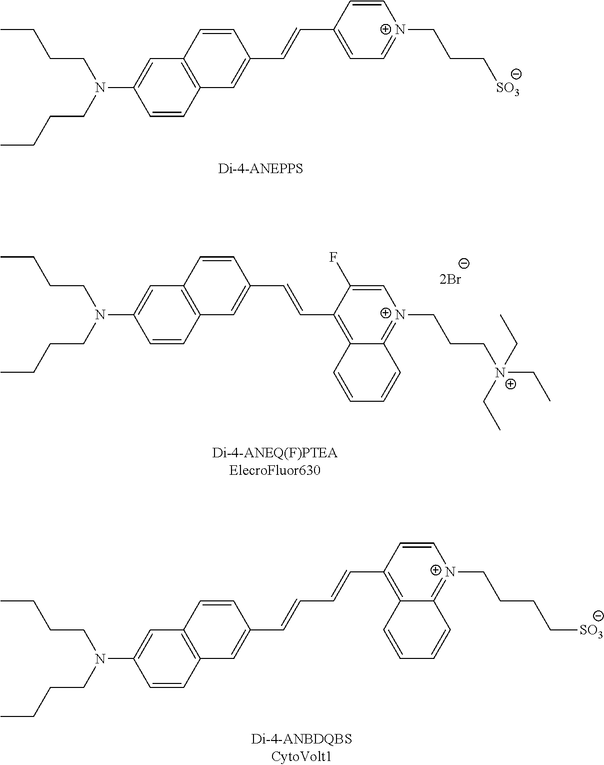

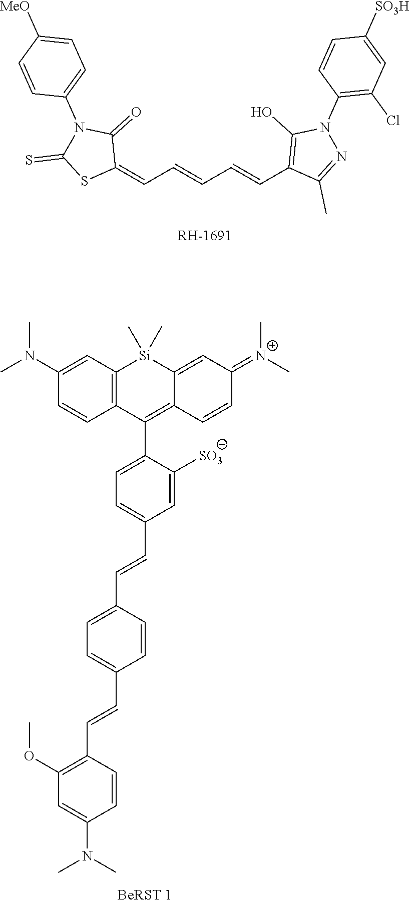

Table 1 summarizes the optical properties of all the newly synthesized VSDs. As expected, the spectra are largely insensitive to alkyl substitution. Table 1 also includes data for ElectroFluor630 (aka Di-4-ANEQ (F) PTEA) for comparison. The absorption maxima of 2a in ethanol (EtOH), aqueous buffer (PBS) and when bound to lipid membranes (multilamellar vesicles, MLV) are red shifted by 77 nm, 63 nm and 63 nm, respectively, relative to ElectroFluor630, which is isostructural to 2a. The fluorescence quantum efficiency of membrane bound 2a is not diminished despite the significant bathochromic shift. The absorption and emission spectra of 2a, 2b, and 2c are all shifted to the red by 50-65 nm relative to ElectroFluor630. Similar red shifts are found for 1a, 1b and 1c, when compared to Di-4-ANEPPS.

TABLE 1
Optical Properties of Chromene-Based VSDs.

| | ${λ}_{max}^{abs} ( nm )$ | ${λ}_{max}^{em} ( nm ) ;$ | Voltage Sensitivity ΔF/F per 100 mV; |
|---|---|---|---|
| | EtOH; PBS; MLV | FQE^{a} | Ex/Em (nm)^{b} |
| 1a | 577; 546; 540 | 673; 0.31 | 12%; 660/>745 |
| 1b | 580; 552; 540 | 677; 0.25 | 8%; 640/>695 |
| 1c | 579; 515; 534 | 675; 0.22 | 11%; 630/>745 |
| Di-4-ANEPPS | 496; 447; 464 | 617; 0.30 | 10%; 550/>610 |
| 2a | 701; 633; 614 | 739; 0.10 | 17%; 730/ >780 |
| 2b | 700; 628; 611 | 736; 0.10 | 10%; 720/>780 |
| 2c | 696; 597; 599 | 734; 0.10 | 12%; 730/>780 |
| ElectroFluor630 | 618; 565; 547 | 686; 0.10 | 12%; 645/>745 |

^{a}Fluorescence quantum efficiency (FQE) in lipid vesicle suspensions.
^{b}Long pass filter used.

### VII. Voltage Sensitivity to Membrane Potential on Hemispherical Lipid Bilayer

Testing VSDs in a voltage-clamped hemispherical lipid bilayer allows one to determine the sensitivity of the VSDs as a function of excitation wavelength. This system has a Teflon tube filled with KCl and is immersed in a KCl solution in a cuvette. A lipid bubble is blown at the tip of the tube and spontaneously forms a bilayer membrane. The VSD to be tested is introduced on one side of the bilayer, usually in the cuvette. Electrodes in the tube and in the cuvette allow the imposition of a voltage across the membrane and a tungsten lamp coupled to a monochromator allows excitation wavelength dependence of the fluorescence response to voltage steps to be determined. This screening approach has allows for the comparison of the efficacy of newly developed VSDs in a well-controlled, consistent manner and to also determine excitation and emission wavelengths with optimal.

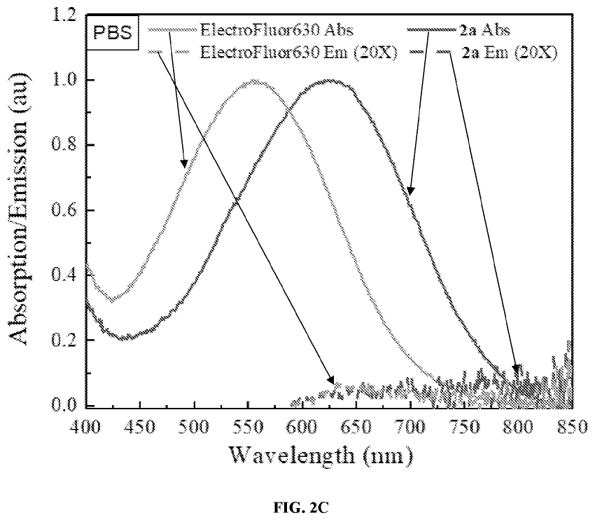

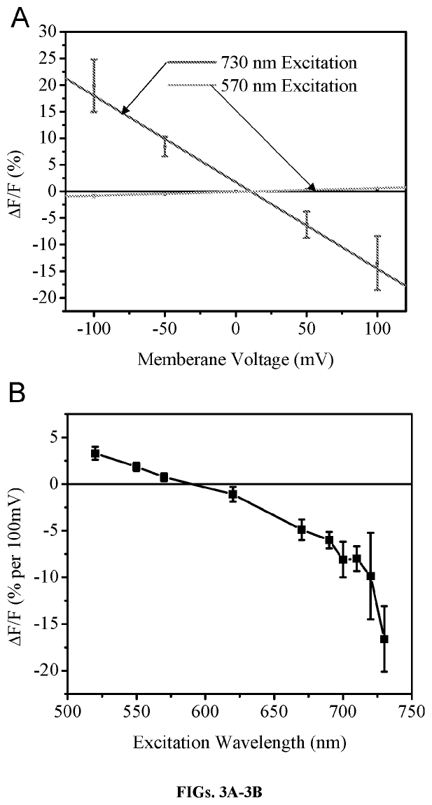

Voltage sensitivities of 2a as a function of voltage is shown in FIG. 3A for excitation at 730 nm and 570 nm. The voltage response is linear over the range of −100 mV to +100 mV; 570 nm is slightly to the blue of the isosbestic point, accounting for the weak but inverted sensitivity at this wavelength. The response of this VSD at different excitation wavelengths is shown in FIG. 3B. It shows typical behavior for an electrochromic VSD: the best sensitivity is detected at the red edge, and the polarity of the response is reversed when excitation is on the blue side of the absorption peak, indicating a voltage dependent blue-shift of the excitation spectrum upon membrane depolarization. This opposite polarity at the blue and red edges underlies the ability to use electrochromic hemicyanine dyes for dual-wavelength ratio imaging. The precise location of the isosbestic point in the excitation wavelength depends on the emission filter used because the emission spectrum is also voltage dependent for hemicyanine dyes.

The magnitude and wavelengths of the optimal voltage sensitivities of all the VSDs on the voltage-clamped hemispherical bilayer are included in the third column of Table 1. The sensitivities of the VSDs containing chromophore 2 are comparable to ElectroFluor630 (last row of Table 1) but the optimal excitation wavelengths for the chromene VSDs are up to 85 nm further to the red.

### VIII. Application to Whole Murine Heart

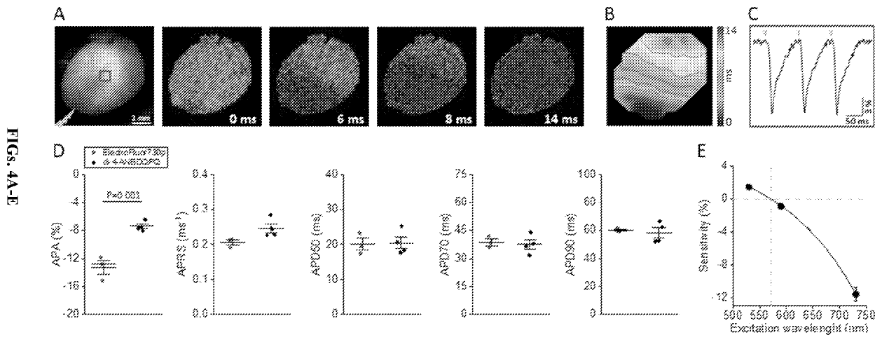

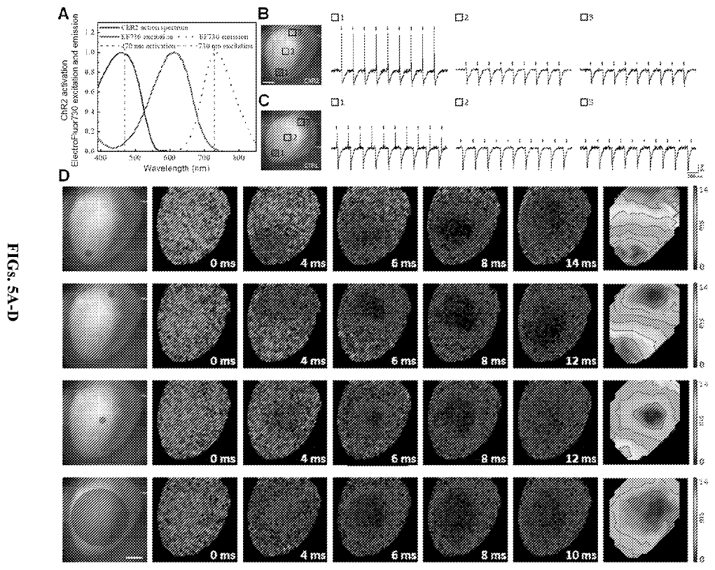

To establish the efficacy of the long wavelength chromene-based VSDs for an important biological application, ElectroFluor730p (2c) was used to optically probe the electrical activity in isolated mouse hearts. using 730 nm excitation. Hearts stained with ElectroFluor730p were electrically paced at the apex with a stimulation frequency of 10 Hz (FIGS. 4A-C). Waves of fluorescently recorded action potentials (AP) could be imaged traversing the heart (FIG. 4A) with propagation across the entire surface in 8 ms (FIG. 4B). FIG. 4C shows that individual APs could be resolved with excellent signal to noise ratios and no signal averaging. AP parameters (AP amplitude: APA; AP rising slope: APRS; AP duration at 50, 70 and 90% of repolarization phase: APD50, APD70, APD90) obtained with the ElectroFluor730p were compared with those obtained with a VSD that has been well established for cardiac applications, di-4-ANBDQPQ (aka CytoVolt2, optimally excited at 630 nm). The sensitivity of ElectroFluor730p to an AP (APA) was higher than that of di-4-ANBDQPQ (FIG. 4D, first panel), measured as a % decrease in fluorescence at the peak of an AP. However, no statistically significant differences in AP kinetics were found (FIG. 4D panels 2-5), indicating that there are no measurable pharmacological effects of the new VSD. Lastly, in FIG. 4E, the sensitivity of ElectroFluor730p (ΔF/F upon 100 mV) was compared at three different excitation wavelengths: 730, 590 and 530 nm. An inversion point (isosbestic point) at ≈571 nm was observed (FIG. 4E). Although FIG. 4E has fewer points, the pattern is similar to the results on the hemispherical bilayer (FIG. 3B). As noted there, this inversion of the response polarity indicates that these chromene-based VSDs can be used in cardiac applications requiring dual-wavelength ratio imaging. In FIG. 4, blebistatin was used to inhibit contractility in the ex vivo murine but ratio imaging would be valuable in beating hearts, where ratiometric measurements can eliminate motion artifacts in the optical recordings.

To demonstrate how 2 optical techniques can be combined in cardiac applications with ElectroFluor730p, 3 ms light pulses of blue light were used (470 nm, 4.0 mW/mm²) to optically induce APs in hearts isolated from transgenic mice expressing ChR2. FIG. 5A shows the overlaid excitation spectra of ChR2 and excitation and emission spectra of ElectroFluor730p. The crosstalk between ElectroFluor730p and ChR2 is minimized here: excitation wavelength for voltage imaging, 730 nm, is far away from activation spectrum of ChR2, and activation wavelength for ChR2, 470 nm, is almost at the minimum of the excitation spectrum of ElectroFluor730p. FIG. 5B shows how brief 3 ms light flashes focused at the apex entrain reliable APs, optically recorded with 730 nm excitation of ElectroFluor730p from three different sites of the heart. Upward spikes due to direct VSD excitation by 470 nm light were observed exclusively at the stimulation site (ROI 1) but even there the downward APs are completely synchronized with the blue light pulses. FIG. 5C shows a control experiment with a non-transgenic heart. Here APs were spontaneously generated from the sinoatrial node (sinus rhythm) and not paced by blue light. The short upward spikes in region 1 in FIG. 5C are due to direct excitation of the VSD with the blue light but are not synchronized with the action potentials, which means the APs are not entrained by 470 nm light in the absence of ChR2. FIG. 5D shows the all-optical approach to manipulate and monitor AP propagation in mouse hearts. Optogenetic stimulation at different sites of a mouse heart (at the apex, at the base, in the middle, and in whole ventricle) using 470 nm blue light and VSD imaging using 730 nm NIR light allows us to induce and monitor AP propagation patterns that are not native to hearts. The channelrhodpsin family of optogenetic actuators, including ChR2, have broad absorbance spectra; although optimal excitation is at ˜470 nm, these actuators can be activated out to 550 nm. As further explained below; the new long-wavelength VSDs introduced here will expand the number of optical recording approaches that will be compatible with optogenetic actuators.

### IX. Discussion

A 7-amino-(2H-chromene-3-yl) scaffold was used as a donor in push-pull hemicyanine chromophores to construct the VSDs. The synthesis overcomes the intrinsic instability of the electron rich amino-chromene by devising a pathway that always maintains it in conjugation with electron withdrawing substituents. The new VSDs have a combination of 2 chromophores using pyridinium and quinolinium acceptors combined with 3 sets of alkyl tails to confer different aqueous solubility and lipophilicity properties for varying biological applications. The absorption and emission spectra (FIG. 3, Table 1.) are red-shifted by 60-80 nm relative to the iso-structural hemicyanines containing aminonaphathalene donors with either pyridinium or quinolinium acceptors but they have similar fluorescence quantum efficiencies. Chromophore modifications to produce red shifts (e.g. extending conjugation with additional double bonds between the donor and acceptor) tend to cause a decrease in fluorescence quantum efficiency. The red shifts afforded by the aminochromene is much larger than that produced by simply extending the chromophore conjugation.

The voltage sensitivities of the fluorescence for all the VSDs using a voltage-clamped hemispherical lipid bilayer system was determined (Table 1.). The voltage sensitivities were comparable to those of their isostructural aminonaphthyl analogs; however, the optimal ΔF/F was obtained at ˜100 nm longer wavelengths. As with the previous generations of hemicyanine VSDs, the fluorescence of the membrane bound molecules is much brighter than the fluorescence of molecules in aqueous media. This is advantageous in biological applications because the background fluorescence is minimized; in some experimental situations, it may be possible to avoid washing away excess VSD and thereby allow fresh dye to replace any bleached molecules in the membrane.

There is a tradeoff of high voltage sensitivity at the red spectrum edge vs. lower fluorescence. For ElectroFluor730 (2a), ΔF/F/100 mV improves with increasing excitation wavelength. Similarly, excitation centered at 630 nm generally yields good results for ElectroFluor630 even if higher ΔF/F may be attained at 645 nm (last row of Table 1). In general, a suitable wavelength band for excitation is at the red edge of the absorbance spectrum; how far toward the edge depends on how photon-limited the experiment is.

One of the VSDs, ElectroFluor730p (2c), has been applied to image action potential propagation in whole murine hearts using 730 nm excitation, by far the longest wavelength used in voltage imaging of fast events like action potentials. It reports both the shape and propagation of action potentials with high sensitivity and high kinetic fidelity. Expanding VSD imaging into this long wavelength region not only allows deeper penetration by minimizing light scattering and absorbance by endogenous chromophores such as hemoglobin, but also offers greater flexibility for multisensor imaging (e.g. voltage and calcium) combined with optogenetics experiments. Furthermore, these VSDs show opposite polarity responses to membrane potential at blue and red edges of their spectra, as do other hemicyanine VSDs. This enables dual wavelength ratio imaging to normalize away artifacts in fluorescent sensor recordings due to bleaching, uneven staining and motion. Eliminating motion artifacts is useful for imaging the beating heart. Existing dual-wavelength excitation VSDs all have short wavelength excitation bands that overlap with the action spectrum of the channel rhodopsin family of optogenetic actuators, which generally stretch from 410 nm-550 nm. The described compounds are examples of a new class of aminochromene VSDs.

### X. Further Discussion

### Research Strategy

### 1. Significance

Electrically excitable cells in the brain and heart use rapid changes in membrane potential as the primary intercellular propagating signal. In the heart, these action potentials (APs) trigger the release of calcium controlling the contraction of cardiomyocytes. Inherited mutations of important ion channels, channelopathies, can alter cardiac APs, leading to disease. In cardiac safety and toxicology, drug developers look for unintended changes in AP waveforms that could lead to serious negative outcomes in patients. Thus, the ability to measure electrical activity in cardiac cells is of great importance in both the laboratory and the clinic. Classical electrophysiology utilizes patch clamp techniques, and remains a critical tool for researchers, providing incredibly detailed data. However, patch clamping is labor intensive and can typically record from only one cell at a time. It can directly probe the molecular basis for channelopathies, but cannot be used to study how they affect heart rhythms, which requires a multisite recording strategy. Likewise, cardiac safety screening, currently involving thousands of compounds per week, is difficult with patch clamp.

Optical approaches provide multisite, parallel recording necessary to overcome the limitations of classical electrophysiology. With a voltage-sensitive dye (VSD), cells in a petri dish or throughout a tissue can be stained and the fluorescence emitted can be detected with cameras for massively parallel recordings because each pixel serves as an optical electrode. In a live animal under open heart surgery, electrical propagation across the heart can be recorded during normal heart rhythms and as the heart transitions to an arrhythmia.

For cardiac recordings, the 3 main challenges for optical electrophysiology are 1. sensor sensitivity and signal-to-noise ratio, 2. motion artifacts since heart cells move when they contract, and 3. pharmacological effects or photodynamic damage. The goal of this proposal is to address the challenges listed above with a new generation of fluorinated hemicyanine VSDs, which we call ElectroFluors™.

In Aim 1 we will continue the development of ElectroFluors™ for various cardiac applications. We will incorporate a novel chromophore that we recently discovered (patent pending) that allowed us to demonstrate the longest wavelength voltage-sensitive dye currently available. Potentiometric Probes incorporates the experience of The Loew laboratory, which has been rationally designing fast voltage sensitive dyes (VSDs) for 40 years. Di-4-ANEPPS, one of the early VSDs to emerge from the lab (Fluhler, Burnham, & Loew; 1985; Hassner, Birnbaum, & Loew; 1984; Loew et al., 1992), became the gold standard for optical recording from cardiac preparations. The ElectroFluors™ are the products of this long and deep experience in VSD development. They are patented fluorinated hemicyanine VSDs originally described in 2012 (Yan et al. 2012).

In Aims 2 and 3 we will validate the optical properties and test for unwanted effects or “toxicity” of new ElectroFluors™ in cardiac applications. We will focus on the cardiac safety screening, (see also Commercialization Plan Market Segment 1). Cardiac toxicology assay is a necessary step in all drug development programs, for any disease target, to prevent the release of any drug with cardiac side effects. Classically this work has centered on detecting drug interactions with the hERG potassium channel, an important channel regulating cardiac action potential waveforms and arrhythmia prevention. New work by FDA (see “CiPA” in Commercialization Plan) and industry partners looks to move this screening work away from heterologous channel expression in cell lines like HEK or CHO, to human stem-cell-derived cardiomyocyte cell cultures or microtissues. By using human cells expressing close to the full complement of human cardiac ion channels, the hope is to make screening more efficient, and more predictive. As mentioned in the letter from Tara Biosystems, many drugs fail current safety screening protocols even when they present no risk to patients, and can therefore not be developed further. We will provide the best VSDs for optical measurements from these human iPSC-CMs with the best combination of ease of use, sensitivity, brightness, lack of toxicity, and ratiometric imaging capabilities, which we describe below. In such a screening environment, it is crucial that the probe itself does not block channels of interest or interfere in any way with normal iPSC-CM physiology. Validating that our fluorinated VSDs are compatible with human iPSC-CMs is a main goal of Aim 2.

Our classic VSD di-4-ANEPPS has already been proposed for dual-wavelength ratiometric drug toxicity screening using hiPSC-CMs (Hortigon-Vinagre, M. P., et al., 2021). We are certain that the ElectroFluors™ to emerge from Aims 1 and 2 will be vastly superior and Aim 3 proposes to establish this. We estimate that 100 thousand compounds will need to be tested each year and could increase as our VSDs accelerate the testing pipeline (see Commercialization Plan). More predictive results from these cellular level screens, will decrease the need for expensive animal studies, such as Guinea Pig heart Langendorff assays (Kilfoil et al. 2021).

An important alternative technology (see Commercialization Plan) to consider is Genetically Encoded Voltage Indicators (GEVIs). GEVIs play a vital role in neuroscience research (Knöpfel & Song, 2019) and are also being applied to cardiac research (Kaestner et al., 2015). The ability to genetically target these protein indicators to specific cell types allows researchers to address many questions not possible with other approaches including VSDs, which bind membranes non-specifically. With tracking neuronal spiking as a main driving force in their development, modern GEVIs have fast response times (Hochbaum et al., 2014; Piatkevich et al., 2018; Villette et al., 2019). There are also ratiometric GEVIs, which sense voltage through FRET between two linked fluorescent proteins (Knöpfel, Tomita, Shimazaki, & Sakai, 2003) or by coupling a non-voltage sensitive second fluorescent protein to a conventional, but spectrally-separate GEVI (Kim, et al., 2022); but these are relatively slow and/or insensitive. Also, as is the case for any fluorescent protein technology, low expression levels may limit the brightness, and therefore signal to noise, when compared to organic dye staining. We believe that our VSDs will be more readily adopted for the cardiac toxicology field because of their convenience, instantaneous response, high sensitivity, and ratiometric capabilities.

The long history of rational design, synthesis, and application of VSDs in the Loew lab and, in particular, the recent development of the ElectroFluors™ justifies our choice of Direct-to-Phase 2 for this proposal. Potentiometric Probes, LLC is dedicated to continuing this history of innovation and service to the optical voltage recording community by building on the expertise established in the academic laboratories of its founders. Indeed, the founding of Potentiometric Probes, LLC was predicated on the realization that continuing to serve the research community through an academic lab has become untenable. We offer the ElectroFluors™ and our classic VSDs at an affordable cost or even gratis for collaborative projects or when labs are between grants. The success of this grant application will assure that we can continue to serve the academic research community by supplying the specialized VSDs required in their many diverse and growing optical recording studies. It will then allow us to firmly establish Potentiometric Probes in the drug testing market, to assure long-term sustainability beyond the seed support requested in this application.

### 2. Innovation

The class of VSDs that we propose to continue to develop are called “push-pull” or “donor-acceptor” hemicyanines. These chromophores are rationally designed with molecular orbital calculations to maximize the molecular Stark effect (aka electrochromism) (Loew, Scully, Simpson, & Waggoner, 1979; Loew & Simpson, 1981).

Because this mechanism operates by directly perturbing the energy difference between the ground and excited state electron distributions in the chromophore, it has 2 key advantages: the response to voltage change is instantaneous and it results in a spectral shift. This means, respectively, that the response will faithfully reproduce the shape of an action potential, and that a dual-wavelength ratio method is enabled to normalize away artifacts due to motion, uneven staining or photobleaching.

The idea of dual-wavelength ratiometry is explained with the help of FIG. 1, which was generated from a real excitation spectrum of one of our dyes, but is meant to represent a generic excitation or emission spectrum where the depolarized spectrum is displaced by 5 nm from the polarized spectrum. The optimal ΔF/F will occur when both excitation and emission are recorded at the wings of the spectrum, where AF is still large, but F is relatively low. The key point is that the voltage-dependent change in intensity will be in opposite directions at the red and blue wings; however, an artefactual change in intensity due to motion or slow bleaching, etc. will affect the intensity proportionally the same at all wavelengths. Therefore, a dual-wavelength ratio can eliminate all the artefactual fluctuations, while enhancing the voltage-dependent fluorescent signal (Montana, Farkas, & Loew, 1989). While this idea is not new and has been applied routinely in imaging calcium, it has become truly practical for voltage upon the development of our new ElectroFluor™ VSDs, most notably in the live in-vivo pig heart (Lee, et al., 2019; see support letter from Dr. Filgueiras Rama), which directly compared ratio imaging with an ElectroFluor™ to an earlier analog without a fluorine substituent.

We now turn to the issue of achieving sufficient signal to noise (S:N), which is a major challenge for fast events like action potentials and ultimately is limited by the number of photons that are emitted (shot noise). To increase the number of photons emitted, one can simply increase the intensity of the exciting light source. The photobleaching rate is proportional to the excitation rate, with a proportionality constant that is dependent on the dye photostability. However, the same excitation rate (and therefore the same bleaching rate) can be achieved with an intense source at the edge of the excitation spectrum as with a weaker source at the peak of the excitation spectrum. FIG. 1 demonstrates, therefore, that it is always best to excite at the edge of the excitation spectrum whether for dual-wavelength ratiometric recording or the simpler single wavelength ΔF/F measurements. The availability of intense stable laser or LED sources can permit much greater flexibility in the choice of wavelengths to optimize ΔF/F without sacrificing S:N. Our strategy for the next generation of dyes is to improve photostability to make it possible to use narrow band excitation to optimize the sensitivity without sacrificing S:N and also permit longer term experiments.

Our rational strategy (Yan, P. et al., 2012) for enhanced photostability involves adding electronegative fluorine substituents to the chromophores to lower the energy of the π-system electrons. Lowering the excited state energy will retard photooxidation and lowering the ground state energy will retard direct oxidation by reactive oxygen species. Relatedly, lowering the energy of the excited state will retard the sensitization of singlet oxygen species and thus reduce phototoxicity (aka photodynamic damage). This is the reason, not often understood, that fluorescein is so much less photostable than rhodamine; the 2 chromophores actually have isoelectronic π-systems, but the overall positive charge in the rhodamine molecules lowers the energy of their electrons relative to the overall negative charge of the fluorescein-based chromophores. Fluorine substitution also generally improves the fluorescence quantum efficiency. Additionally, positioning the electronegative fluorines at various points on the hemicyanine can tune the spectra to higher or lower wavelengths (Yan et al., 2012). As in the past, we will be guided by molecular orbital calculations to optimize the large charge redistributions that underlie the voltage sensitivity of the hemicyanine dyes. We have decided to market the entire class of fluorinated hemicyanine dyes in our catalog as ElectroFluor™; an appropriate suffix will identify the optimal excitation wavelength and any other physical attributes such as high solubility or long persistence.

Regarding these physical properties, we will again be guided by rational strategies. For example, we recently synthesized ElectroFluor-630p™ (Di-5-ANEQ(F)PTEA) in response to a customer's requirement for persistent staining in long term experiments (support letter from Dr. Rogers). Deep tissue penetration (Fedorov, V. V., et al., 2011) or high VSD solubility for intracellular application (Acker, C. D., et al., 2011) may also be achieved by appropriate modifications of VSD side chains without modifying the electrochromic chromophore. On the other hand, chromophore modifications may be needed for experiments that combine VSDs with other optical technologies, such as calcium sensing (e.g. Lee et al., 2012; see support letter from Peter Lee) or optogenetic activation (Scardigli, M., et al., 2018; see support letter from Leonardo Sacconi); this is to avoid artifacts in the measurements due to overlapping spectral windows—especially challenging for dual-wavelength ratiometry. Long-wavelength VSDs are also optimal for avoiding interference by hemoglobin in blood-perfused (Matiukas. A., et al. 2007) and in vivo preparations (Lee, et al. 2019; see letter of support from Dr. Filgueiras Rama); additionally, the new ElectroFluors™ with even longer wavelength spectra that we introduce below will reduce light scattering associated with thick tissue imaging. Thus, to support the cardiac research community, we propose to expand the Potentiometric Probes catalog with new fully characterized and tested ElectroFluors™.

### 3. Approach

The approach in this Direct-to-Phase II proposal is a continuation of our extensive experience with designing voltage-sensitive dyes, testing their properties, and advising users to get the most out of their recordings at minimal expense. It is based on collaboration with Cytocybernetics, Tara Biosystems, Essel R&D, Photoswitch Biosciences, IonOptix, and academic collaborators (see letters of support). The high-quality imaging data with high signal-to-noise from iPSC-CM cell culture and microtissues demonstrate feasibility and proof-of-concept, the stated goal of a phase I SBIR. Additionally, Potentiometric Probes succeeds the long record of VSD development at U. Connecticut, including the patented fluorinated hemicyanine dyes (Yan, P. et al., 2012; U.S. Pat. No. 8,993,258, 2015) that form the basis for the ElectroFluor™ VSDs. This Phase II project is thus a natural extension towards commercialization goals. We will demonstrate the efficacy of ElectroFluor™ VSDs for high quality optical recording in cardiac tissue and human iPSC-CMs.

Preliminary Results supporting Direct-to-Phase II. We are excited to focus this direct-to-phase 2 application on expanding our catalog of ElectroFluor™ VSDs and obtaining the validation data for cardiac applications necessary for our commercialization efforts in these spaces. Over the past 3 years we have worked with collaborators at UConn and elsewhere to begin this validation work in cardiac preparations to test the hypothesis that ElectroFluor™ VSDs are the best tool for optical voltage recordings in cardiac cell culture, tissue, and in-vivo preparations. Below we present highlights from a great deal of data collected in-house as well as in the labs of collaborators, equivalent to the successful outcome of a Phase I SBIR grant. As mentioned above, we recently synthesized ElectroFluor630p™, a new VSD that will shortly enter our catalog.

We were motivated by separate requests from a company, Tara Biosystems, and the academic lab of Dr. Jack Rogers (see letters of support), for cardiac applications requiring recording over the course of longer than a full day. This will require dyes with both improved persistence (i.e. long washout times) and photostability (to allow for recording over long periods without appreciable bleaching or phototoxicity). We addressed these requirements via synthesis of a fluorinated chromophore (to improve photostability) with lengthened alkyl chains (to improve the strength of membrane binding and therefore persistence); we are calling it ElectroFluor630p™ (630 for the optimal excitation wavelength and p for persistent). ElectroFluor630p™ is similar to ElectroFluor630™, except that the butyl chains have been replaced with pentyl chains. We characterized this new VSD on both our voltage clamped hemispherical bilayer and on AtT20 cells. It shows excellent sensitivity with opposite polarity for blue-vs. red-edge excitation (+5.2 and −19.4% ΔF/F/100 mV, respectively) as shown in FIG. 2. These data serve as both preliminary results for Aims 1 and 2 and an example of the kinds of data we routinely gather to test our VSDs. When converted to ratio measurements where data collected with blue excitation is divided by that collected with red excitation and converted to percent, these data yield a sensitivity of 29.3% ΔR/R/100 mV, already better than our biggest competitor dye (˜25% ΔF/F/100 mV, Miller et al. 2012). In addition, we will follow-up on these measurements extending the excitation wavelengths to include 660 nm. This is expected to further increase sensitivity and is an important wavelength for commercialization since the Photoswitch Bolt system, which is being sold for high-throughput cardiac safety screening studies (see letter from Stephen Smith), uses 660 nm laser excitation.

A newly synthesized patent-pending class of chromophores developed in the last year, extends the VSD measurements ˜100 nm further to the red than had been previously possible, with a demonstrated optimal sensitivity at 730 nm excitation. This has been achieved by incorporating a chromene electron donor moiety in the place of the naphthalene in the existing ElectroFluors™ (FIG. 3). Importantly, despite the large spectral red-shift, the chromophores are isostructural and the fluorescence quantum efficiencies are not diminished;

this means that the solubility/persistence and brightness will not be compromised by the extended conjugation typically employed to lengthen wavelengths. And because they are electrochromic, they can be used ratiometrically: while ElectroFluor730™ has a ΔF/F/100 mV of −17% for 730 nm excitation (FIG. 3), it shows +3% at 560 nm. This wavelength pair is ideal for dual wavelength ratio imaging of voltage simultaneous with optogenetic actuation of CheRiff or ChR2, neither of which excite at >550 nm. Our first results in a biological preparation was obtained 2 weeks ago from our collaborator Dr. Sacconi (FIG. 4). Importantly, the hemicyanine chromophore backbone for this new series of near-infrared ElectroFluors™ has not previously been reported to our knowledge (searched using Google and both the SciFinder and Reaxsys databases; our patent attorney has also been unable to find prior art). This chromophore will for the first time allow ratiometric voltage recording with simultaneous optogenetic actuation.

Turning to Preliminary Results for Aim 2. we have already described our characterization of a new VSD in patch clamped tissue culture cells in FIG. 2.

ElectroFluor-560™ and ElectroFluor-630™ have been successfully applied to cardiac applications. The dual wavelength ratiometry produces high fidelity optical APs by normalizing away motion artifacts, a key advantage of our approach toward characterizing APs in healthy contracting preparations including iPSC-CM cell cultures and microtissues. The ability of ratiometric recording methods to eliminate motion artifacts is most striking in in-vivo preparations and we highlight a spectacular study with ElectroFluor-630™ (FIG. 5) showing recordings from an in-vivo open-heart pig. Note especially the high fidelity cardiac action potentials recovered by taking the ratio of the fluorescence recorded at 2 excitation wavelengths; the APs are completely obscured by motion in the individual blue and red fluorescence traces. The paper that describes this collaboration (Lee, et al., 2019; letter of support from Dr. Filgueiras Rama) has movies of electrical waves during sinus rhythms and arrhythmias in the contracting heart; we encourage reviewers to check them out.

Directly relevant to Aim 3, through collaborations with Tara Biosystems (see letter of support), Potentiometric Probes provided guidance in implementing VSD imaging for a cardiac safety assessment based on contractility measurements of engineered cardiac microtissue (FIG. 6A). High quality ratiometric VSD measurements of drug-induced changes in AP waveform were possible (FIG. 6B). Simultaneous contractility measurements are central to their engineered cardiac microtissue (ECT) safety assessment assay (FIG. 6C), necessitating dual-wavelength ratiometry for accurate AP recording. In Aim 3, we will test the best ElectroFluors™ to emerge from Aims 1 and 2 for their suitability in Comprehensive In-Vitro Proarrhythmia Assays (CiPA).

The above may be summarized in the form of Phase I equivalent milestones:
Establish compatibility of electrochromic voltage-sensitive dyes for ratiometric voltage imaging in cardiac preparations: we performed optical voltage recordings and measurements of AP durations (APD) in 3 cardiac preparations (human iPSC-CMs cell culture, microtissues, and in-vivo pig) using 3 ElectroFluors™ (FIGS. 2, 4-6).
Design and synthesized new electrochromic dyes for cardiac applications. ElectroFluor-630p™ designed and tested for improved persistence and prolonged in-vivo whole-heart electrical recordings. (FIG. 2, see letter from Rogers). Rationally designed and synthesized a completely novel electrochromic chromophore to expand the range of ElectroFluor™ VSDs into the near infrared (FIGS. 3, 4).

### Aim 1. Synthesize New Ratiometric Cardiac VSDs Tailored to Specific Cardiac Tissue and Organ Imaging Experimental Needs

Rationale. This aim proposes to expand our catalog of VSDs focused on cardiac applications. We will develop new dyes from the existing styryl/hemicyanine chromophore backbones with substitution patterns that will meet the requirements (spectral window, solubility, membrane affinity, ionic charge) of specific applications. Of course, improved sensitivity will always also be a goal. We are constantly engaged in extensive email or telephone exchanges with our customers to help them choose the best VSDs for their applications and to troubleshoot how to optimize their measurements. In the process, we are accumulating a deeper understanding of the range of applications and biological preparations that the optical recording community employs, allowing us to anticipate new markets for new optimized dyes.

Another priority will be the development of long-wavelength VSDs that are suitable for dual-wavelength ratio measurements and compatible with optogenetic actuators; the best optogenetic actuators are excited with blue light, but have spectra that tail to ˜550 nm. The longest wavelength dye in our current catalog. ElectroFluor630™, has optimal dual excitation at 470 nm and 630 nm (FIG. 6), so the lower wavelength would produce some unwanted activation of the light-sensitive channels. Our collaboration with Dr. Sacconi (Crocini, C., et al., 2016; Scardigli. M. et.al., 2018; see letter of support) on optogenetic control of cardiac rhythms with simultaneous VSD recording has been limited to single wavelength recording at 630 nm, using blebistatin to control motion artifacts pharmacologically-clearly not ideal, especially if contraction strength also need to be investigated. Thus, our plans include methods to engineer chromophores with spectra shifted by at least 100 nm to the red.

Chemists typically design longer wavelength chromophores by extending the conjugated π-system. For the hemicyanines, we have achieved this by using naphthylamine (instead of aniline) donor moieties and quinolinium acceptors (instead of pyridinium); incrementing the number of double bonds in the linker moieties produces additional red shifts. However, increasing the size of the linker can reduce fluorescence quantum efficiency, solubility, and also photostability. Therefore, we have judiciously employed fluorine substituents to red shift the spectra without further increases in the size of the π-system (Yan, et al., 2012); indeed, fluorination intrinsically also imparts improved photostability to the ElectroFluors™. The synthetic strategy for extended conjugation and fluorination is discussed below in relation to Scheme 1, below. The longest wavelength VSD in our catalog is ElectroFluor-630™, but as shown in FIG. 3 we have recently developed a completely new chromphore with optimal voltage-sensitive wavelengths that are red shifted by ˜100 nm compared to isostructural existing ElectroFluors™, preserving quantum efficiency and solubility. This Aim will further develop this new chromene-based class of VSDs.

Long wavelength excitation will also have the advantage of permitting deeper light penetration into thick preparations, because of the inverse 4th power dependence of light scattering on wavelength, and the absence of endogenous chromophores with absorbance spectra in that region. The key message is that a comprehensive catalog of VSDs will serve the academic and commercial optical recording communities in ways that our small company is uniquely equipped to achieve.

Research Strategy. We will rationally synthesize and characterize hemicyanine VSDs variants with a range of wavelengths, solubilities, staining persistence and ionic charges. The range of combinations is illustrated in Scheme 1. We have experience with the synthesis of the starting materials in Scheme 1 as well as the aldol condensation reactions of the common final step (Hassner, Bimbaum, & Loew, 1984; Wuskell et al., 2006; Yan et al., 2012). Considering all the combinatorial possibilities, including the 8 combinations of the 3 fluorine substitutions (R′), there are 1,536 VSDs that could emerge from this library of starting materials. We therefore outline rational molecular engineering considerations that will focus our efforts, always being driven by market demand as well as the concurrent testing described in Aim 2.

Fluorine substitution is very desirable because of its photostability benefits, but it also controls the wavelength of the excitation and emission spectra (Yan et al., 2012). These spectral windows are also controlled by the choice of pyridinium or quinolinium moieties (1 in Scheme 1) and the number of double bonds in the linkers (n). Our current catalog of VSDs permits customers to choose dyes for optimal dual-wavelength excitation anywhere within a

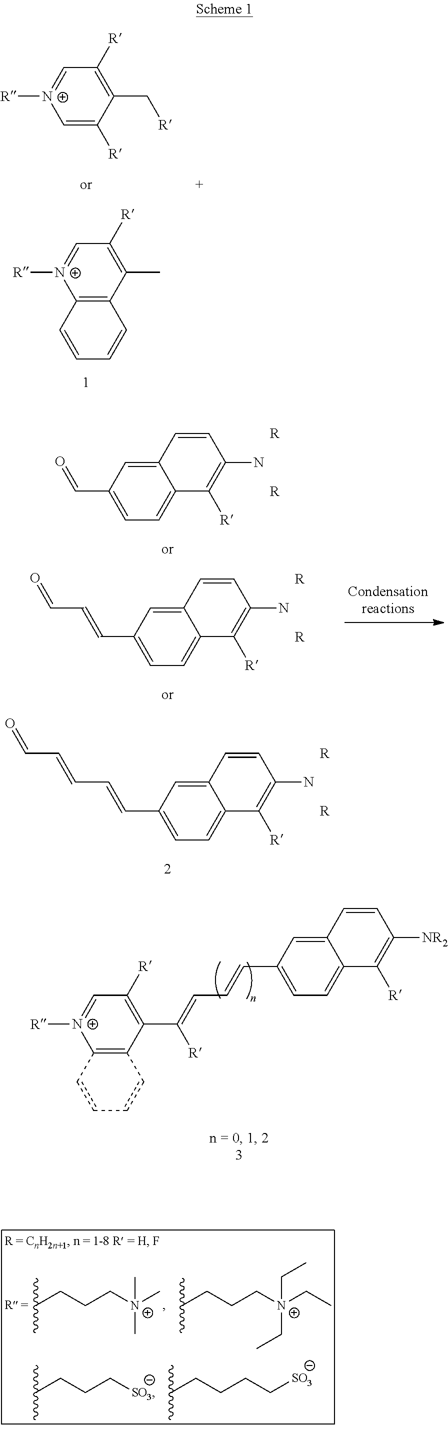

range of ˜410-490 nm and 520-630 nm. Fluorine substitution on the quinolinium (R′=F) and extending the linker length will red-shift the spectra. The combinatorial variety of chromophores will allow us to meet the precise wavelength requirements for combination with other fluorescent indicators (Lee et al., 2012) or avoidance of blood absorbance (Matiukas et al., 2007) that our customers may demand. We will also develop fluorinated derivatives of our highly successful near infrared VSDs based on the ANBDQ chromophore (di-4-ANBDQPQ and di-4-ANBDQBS) (Fedorov et al., 2011; Fedorov et al., 2010; Kee, Wuskell, et al., 2008; Lee et al., 2011; Matiukas et al., 2007; Warren et al., 2010; Wuskell et al. 2006; Zhou, et al., 2008), which are currently being marketed through our collaboration with CytoCybernetics as CytoVolt1 and Cytovolt2, respectively. ElectroFluor630™ is an example of such a new fluorinated VSD; it has shown significant advantages in terms of sensitivity, stability, and minimal phototoxicity in the translational in-vivo pig model of FIG. 6 (Lee, et al., 2019; see letter of support from Dr. Filgueiras Rama).

The alkyl chains at the amino end of the chromophores (R) control both solubility and membrane persistence. Derivatives with R=butyl are suggested as the default systems because they display reasonable solubility for external staining and moderately long persistence.

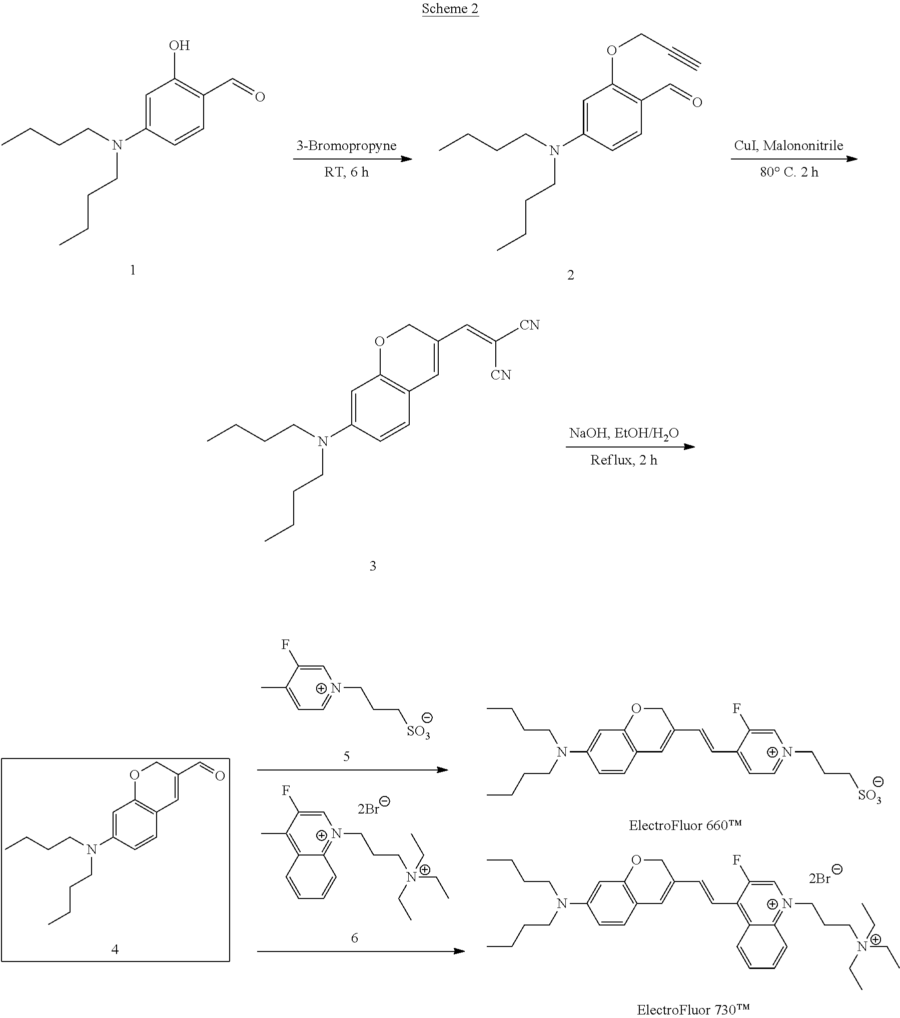

R=ethyl offers VSDs that are highly soluble; they may be used for single cell labeling via internal application through a patch (Acker, Yan, & Loew, 2011) or for very deep tissue penetration (Fedorov et al., 2010). Fine tuning of solubility vs. membrane staining will often require synthesis of dimethyl or dipropyl derivatives. For long term studies where persistence and slow internalization are needed, we will synthesize Di-5 or Di-6 derivatives. For example, at the recent request of Jack Rogers (see his letter of support) we completed the synthesis of ElectroFluor630p™ (Di-5-ANEQ (F) PTEA) to enable him to do longer term studies on pig heart. Our own initial results with this new VSD were described above (FIG. 2).

Solubility and internalization rate may be further modulated using the appropriate headgroup, R″. Our default is the triethylammonium (TEA) headgroup, as it imparts good solubility and slow internalization. The propyl-or butyl-sulfonates (PS and BS, respectively) are the standards for the older classic dyes like Di-4-ANEPPS; their excellent persistence may outweigh their relatively poor solubility in some applications, and they can be delivered with a vehicle like Pluronic F127 (Lojewska & Loew, 1987).

As introduced in the preliminary results, we have prepared new longer wavelength VSDs, based on an aminochromene electron donor moiety. We sought to design a more potent electron donor than the aminonaphthyl of our current ElectroFluors™. The oxygen within the chromene is situated at a position so that its lone pair electrons can reinforce the electron donor amino group (exemplifying rational chromophore design). FIG. 3 provides the key characteristics of ElectroFluor-730™, demonstrating excellent voltage sensitivity at 730 nm, ˜100 nm higher than any previous VSDs. The first results in a cardiac preparation generated by collaborators are shown in FIG. 4. We prepared a second new chromene VSD, ElectroFluor-660™, with excellent voltage sensitivity; this chromophore uses a fluoro-pyridinium acceptor and it actually is shifted by 90 nm when compared to its isostructural aminonaphthyl ElectroFluor™. Scheme 2 details the synthesis of this new class of dyes. The starting materials in Scheme 2 can readily incorporate various hydrophobic anchors and hydrophilic headgroups using the same strategies illustrated in Scheme 1. We believe these are the longest excitation wavelength voltage indicators (VSD or GEVI) able to resolve action potentials. This will allow, for the first time, dual-wavelength ratiometric recording of voltage in conjunction with activation of ChRs, which have optimal excitation at 460 nm but have a long red tail out to 550 nm that can be avoided with ElectroFluuor-730)™. In collaboration with the Sacconi lab (Crocini, et al., 2016; Scardigli, et al., 2018; Credi, et al. 2021; see letter of support from Dr. Sacconi), this idea will be tested.

As a matter of routine, we determine the absorbance and fluorescence spectra of all our newly synthesized dyes in ethanol, saline, and lipid vesicles; we then characterize them on a voltage clamped hemispherical lipid bilayer (HLB) system (see Loew et al., 1979 for the original and Yan, Acker and Loew, 2018 for the latest version of this system), which allows us to determine the wavelength dependence of the fluorescence sensitivity (i.e. ΔF/F per 100 mV) as well as the kinetics of the response to a voltage clamp pulse.

Potential Problems and Alternative Approaches. With regard to dye stability, extending the linker length may be problematic. We have experience with extending the conjugation using alternate linkers including furan, thiophene and squaraine (Wuskell et al., 2006; Yan, Xie, Wei, & Loew, 2008); additionally, the chromene based dyes will obviate these issues by increasing wavelength without lengthening the linkers. VSDs with R′ longer than butyl and/or R″=PS or BS, may be relatively insoluble and difficult to deliver to target cells or tissues. As already noted, we routinely use Pluronic F127 as the vehicle for promoting delivery, as it is non-ionic, non-toxic, and readily available. We have recently had reports from collaborators that they have successfully used Kolliphor® EL to deliver very insoluble VSDs to rabbit and rat cardiac preparations. Another issue is potential pharmacological effects of the VSDs due to interactions with ion channels in the membrane. For example, although none of our customers have reported this, the triethylammonium (TEA) head group may have some effect on K+ channels, because of its similarity to the known blocker tetraethyammonium. For such situations, we can explore the trimethyl ammonium headgroup (TMA), which will retard the flipping rate, yet potentially be less problematic pharmacologically than the TEA head group. With regard to synthesis, we have experience with the chemistry in Schemes 1 and 2 and are confident that we will be successful for the vast majority of our synthetic targets. However, if there are problems, we have experience with the palladium-catalyzed coupling (Heck coupling); in fact, this is how Di-4-ANEPPS was originally synthesized (Hassner et al., 1984), although it is now being prepared via the aldol condensation in Scheme 1. Overall, we feel we have the in-depth understanding of the chemistry of VSDs to meet the needs of the optical recording community.

Metrics. We will benchmark all new dyes against di-4-ANEPPS with regard to fluorescence quantum efficiency, photostability and voltage sensitivity using the HLB system, requiring that they be comparable or better before passing them on to Aim 2. All electrochromic VSDs respond to voltage transients instantaneously, but this will be verified. VSDs are then further characterized and tested in Aim 2; analyzing how the structures determine Aim 2 VSD performance metrics, will feed back on this Aim to prioritize new synthetic targets.

Specific Aim 2, Months 1-24. Characterize Fluorinated Ratiometric VSDs in HEK Cells and iPSC-CMs

Rationale. The scope of this Aim and its tiered structure is meant to be broad enough to allow us to link specific molecular features of dyes to desirable properties such as sensitivity as well as undesirable effects such as AP prolongation. For cardiac safety studies such as CiPA, which focuses on APD recordings pre and post drug application in human iPSC-CMs, it is imperative that the dyes used do not themselves alter APD or spontaneous beating. This careful analysis has not been shown for competitor dyes such as FluoVolt and our preliminary data using FluoVolt (FIG. 7) shows significant effects on APD and spontaneous beating. Thus far, these effects have not been noticeable in recordings with ElectroFluors (FIG. 7). Detailed screening data including undesirable “toxic” side effects, will be available through our website and will serve as a guide for our customers to choose the best VSD for their application and instrumentation.

We present a tiered screening protocol to identify the best VSDs and establish optimal spectral parameters and procedures for staining. We will build upon preliminary data (Phase I equivalent, FIG. 2) and systematically determine optimal wavelength pairs for ratiometric optical electrophysiology for the ElectroFluors™ to be synthesized in Aim 1, using the HLB results as a guide. These studies will also allow us to detect potential toxic effects on cell physiology by the combined action of VSD staining and exposure to exciting light. We will also apply a subset of these methods to assess competitor VSDs for their suitability for iPSC-CM optical electrophysiology. We will not attempt to assess GEVI technologies for genetically targeted expression of voltage sensors in tissues and organs. This technology is complementary rather than competitive with VSDs.

Research Strategy. Based on the hemispherical lipid bilayer measurement in Aim 1, we will take any VSD with a sensitivity of >10%/100 mV at any wavelength and use the optimal ratiometric wavelength pair with HEK cells that have been genetically engineered to spontaneously fire APs (Park et al. 2013). These cells express NaV 1.3 and KIR 2.1 and produce propagating waves of excitation that can be imaged with VSDs (or GEVIs). They offer a convenient screening system to optimize staining procedures and for further optimization of optical parameters including excitation wavelength pairs. Current clamp of these cells will allow us to validate the kinetic response of candidate VSDs, which should be instantaneous based on the electrochromic mechanism (Loew et al., 1985). This screen will also offer the first evidence of any toxic effects on cell physiology, which we can assess based on an alteration in the AP activity as a function of VSD concentration and light exposure. We expect that ˜50% of the VSDs that we test in this spiking HEK cell screen will prove worthy of further investigation in cardiac cells.

Based on previous experience indicating effects on potassium currents at high concentrations, we will then directly test the best dyes to emerge from the above spiking HEK cell screen, specifically for effects on the hERG potassium channel. The goal will be to determine whether or not there is an effect, and if so, to get a sufficiently reliable estimate of IC50, the dye concentration leading to a 50% block of the channel. We will use this data to compare dyes and draw connections between this form of “toxicity” and dye structures—feeding back into our VSD design in Aim 1. Differences smaller than a factor of 2, are not significant and can be ignored. CytoCybernetics offers this test as a service for pharmaceutical clients and has agreed to help with this testing, see letter of support. We have budgeted a study involving sufficient repeat trials and concentrations for 10 dyes for a total of 150 test points. Included in the 10 dyes will be new dyes from Aim 1, including ones with the new TMA head group for comparison to TEA head group dyes. We will also test the new long wavelength ElectroFluors™ developed via Scheme 2. These experiments involve whole cell patch clamp recordings on a HEK293 cell line expressing hERG and are therefore much faster and less expensive than experiments on human stem cell-derived cells, which will be used next.

Experiments on human stem cell-derived cardiomyocytes will be performed both in-house and at Cytocybernetics and TARA Biosystems. Two experiments are available that afford true control measurements in the absence of dyes. First, electrode electrophysiology including current and voltage-clamp can be used with and without the presence of dyes to look for possible effects on: resting membrane potential, action potential rise times (sodium channel dependent), and action potential repolarization and durations (mainly potassium and calcium channel dependent). Concentration curves from the hERG block assay will be used to select initial dye concentrations whenever possible and we have budgeted for 5 concentrations for 10 dyes, 50 data points. Preliminary data from Cytocybernetics (FIG. 8) with simultaneous patch clamp and optical voltage recordings demonstrates perfect correspondence for both HEK293 (FIG. 8A) and hiPSC-CM cells (FIG. 8B).

Cell cultures and microtissues from TARA Biosystems (FIG. 6) will be utilized for simple experiments looking for concentration dependent effects of VSDs on spontaneous beating. We have some evidence (e.g. with FluoVolt in FIG. 7C,D), that spontaneous beating can slow in the presence of dyes, so it is necessary to investigate further in a quantitative way that lets us compare dyes with different chemical motifs. Either IC50s could be measured for the number of beating cells in monolayers, or maximal concentrations leading to significant increase in the interbeat period can be measured for monolayers or microtissues.

The same cell cultures and microtissues can be used for imaging studies to measure concentration dependent changes in action potential duration (APD). The high signal-to-noise allows accurate APD measurements, down to ±1 ms, and should also allow us to reduce the dye concentration significantly and still measure APDs with sufficient accuracy. To look at phototoxicity, we will take select cultures and image them with increasing excitation powers for increasing doses of light. Brightness, photobleaching, and effects on APD can all be measured for quantitative comparisons.

As noted in Aim 1, development of a VSD for ratiometric recording in conjunction with optical pacing will be a high priority. We anticipate that VSDs such as ElectroFluor730™ (Scheme 2, FIG. 3) will allow for low excitation spectrum edge excitation at ˜560 nm, above the activation spectrum of ChR2. The long wavelength edge should be ˜730 nm. We will collaborate with the Sacconi lab to test these dyes in iPSC-CMs as in our recent paper (Credi, et al. 2021), which employed single wavelength excitation of di-4-ANBDQBS (letter from Leonardo Sacconi and FIG. 4 for their first results with ElectroFluor-730p™).

Several VSD technologies fall into the category of high speed sensors like the ones proposed here and are therefore competitors. In general, very few direct head-to-head comparisons have been done. FluoVolt is one of the most recent new VSDs that has generated significant interest in the research community owing to its good sensitivity (Miller et al., 2012); however, when published, the dye was compared to Di-4-ANEPPS rather than more modern dyes available at the time. We tried FluoVolt™ in cardiac microtissues and found roughly comparable (Fluo Volt lower by a factor of 2) signal-to-noise ratio (SNR) and sensitivity (FIG. 7), but noticed significant pharmacological effects, as discussed in relation to FIG. 7. The FluoVolt™ example raises a number of questions, and makes it clear that a more careful comparison of imaging properties (sensitivity, signal-to-noise) along with toxicity effects between dyes is needed. This point has been made to us by researchers and companies (see letter from IonOptix) who have tried FluoVolt™ in their own labs. We will undertake a survey of competitive VSDs similar to the experiments described for FluoVolt. Human iPSC-CM monolayers will be used for optical recordings to compare dyes. Batch-to-batch variability can be considerable with iPSC-CMs and will be taken into account, by choosing a “standard” dye like ElectroFluor560™ and taking measurements on at least some cells from every batch, for control/comparison purposes.

Dyes to be compared (ratiometric yes/no): FluoVolt™ (no), Berst (no), RH1691 (Shoham et al., 1999, no), ANNINE-6plus (Kuhn et al. 2004, yes), as well as our own “classic” dyes Di-4-and Di-8-ANEPPS (yes).

Metrics for imaging performance: sensitivity (ΔF/F/100 mV), optical AP kinetics, signal-to-noise ratio

Markers for toxicity: action potential duration, spontaneous beating

As a result of this aim, we expect to produce a data set comparing different VSDs and their most important features relevant to cardiac imaging. Such a data set, including both imaging properties like sensitivity and aspects of toxicity such as APD prolongation, has not been shown previously. These data are necessary for academic and industry end users to rationally choose the best dye for their application and are especially relevant to our CiPA focused commercialization efforts.

Potential Problems and Alternative Approaches. We and our collaborators have experience with all the methods that we propose in Aim 2 so we do not anticipate technical difficulties. A happy problem that we might anticipate is that there may be too many good candidate VSDs to realistically characterize with the tests that are further down our tiered list of assays. Realistically, we expect to about ˜12 VSDs to fully emerge from our tiered screening.

Metrics. To move down our tiered screening:
Spontaneously firing HEK cells: >10% ΔF/F/AP and <5% decrease in AP kinetics.
Patched HEK293 cell line expressing hERG: >10% ΔF/F/AP and <5% inhibition of hERG.
hiPSC-CM cell cultures and microtissues: >10% ΔF/F/AP, <5% AP distortion, spontaneous beating.
   Specific Aim 3 Months 12-24. Use CiPA Drug Panel to Validate Ability to Capture Drug Effects on AP Waveforms, Spontaneous Beating Using Human iPSC-CMs

Rationale. The success of the Comprehensive In-Vitro Proarrhythmia Assay (CiPA) depends on 1. the ability of human stem cell-derived cardiomyocytes (iPSC-CMs) to respond in a way that mimics or is predictive of responses in human patients when exposed to pharmaco-active substances and drugs and 2. the ability of a recording system to measure these responses accurately, reliably and efficiently. Such “predictive” measurements would allow higher throughput testing of more compounds in this in-vitro setting potentially leading to massive cost savings in cardiac safety screening, far fewer animal studies (Kilfoil et al. 2021), and, ultimately, the approval of safer, more effective drugs. The FDA partnered with pharmaceutical companies, private companies, and contract research organizations (CROs) to test this possibility using a panel of 26 drugs with known risk factors in human patients (Blinova et al., 2017, cipaproject.org). Both microelectrode array systems (MEAs) and optical recordings with the voltage-sensitive dye Di-4-ANEPPS were used to look at AP waveforms before and after drug application at various concentrations. Overall, there was very good agreement between in-vitro action potential duration recordings and known drug effects in patients' EKG recordings and they even found correlations between drug concentrations in-vitro and known patient drug doses.

In this specific aim, we will record action potential durations (APD) in iPSC-CMs and quantify changes including APD prolongation that is a known risk factor for potentially fatal arrhythmias (Torsade de pointes, TdP). We believe validation work reproducing known effects similar to what others have done previously is a necessary step for commercialization of ElectroFluors™ and their adoption by pharma companies and CROs for this application. At the same time, we will implement and validate all of the potential improvements in terms of voltage-sensitive dyes and recording methods and expect to show agreement with expected effects, but at much higher resolution and accuracy than originally reported. Since the original study was done only with Di-4-ANEPPS, at one concentration, it is important to validate that newer voltage-sensitive dyes including fluorinated dyes do not interfere or mask expected drug effects at concentrations that provide adequate signal-to-noise. If high dye concentrations are detected to have effects on hERG channel conductance from Aim 2, then they would not be expected to interfere with hERG channel blockers at smaller concentrations in this aim. The original paper with Di-4-ANEPPS did use ratiometric imaging, by using a single excitation wavelength and splitting the emission between 2 single-pixel detectors for short and long emission wavelengths. Previous studies including phase I work on fluorinated dyes show that dual excitation ratiometric measurements lead to far better sensitivity than dual emission measurements.

Research Strategy. We will choose 4 drugs from the CiPA panel from the 8 that are considered high risk for arrhythmias (Torsade de pointes, TdP) and long QT intervals including Quinidine and Dofetilide and apply them at the concentrations that led to significant effects in the Blinova et al. 2017 data set. Additionally, we will use E-4031, a specific hERG potassium channel blocker. We believe that choosing 5 drugs that are known to prolong QT intervals by acting on differing molecular determinants of action potentials (Quinidine acts primarily on sodium channels, but also on potassium channels, while Dofetilide primarily affects delayed rectifier potassium channels) will convincingly demonstrate that optical recording with our VSDs is appropriate for cardiac safety screening studies using iPSC-CMs. In the process, we will also establish the optimal experimental conditions for these assays with each VSD tested and will pass this data on to our customers.

Optical recordings will be made using a camera-based system as in FIGS. 2, 5 and 7 with spatial resolution similar to that shown in FIG. 2 covering less than 10 cardiomyocytes. Action potential durations along with spontaneous spiking rates will be recorded. Depending on variability in the data, we expect to record from 2-4 separate regions in each dish, for 3-5 separate dishes per compound and dye tested, for a total of 6-20 dishes per combination.

Dyes will be chosen selectively for this Aim since dyes will first be characterized in previous aims for their sensitivities and other properties as described above. Only new chromophores or dyes with untested alkyl chains or head groups will need to be tested.

Based on the optimal parameters for recording with each VSD established in Aims 1 and 2, we will perform at least 5 replicates for each drug study with each dye. Results including plots of mean and standard deviations of electrophysiological parameters as a function of drug concentration will be published along with the spatial, temporal resolution of the recording system and the actual signal-to-noise levels of the recordings.

Completion of this aim will result in a detailed data set that is publishable on our website if not in a peer-review journal demonstrating compatibility of ElectroFluors IM with CiPA-compatible recordings of AP waveforms for quantitative duration (APD) measurements and known drug effects. Using several ElectroFluors, we will generate concentration-dependent APD curves necessary for CiPA. Drug effects and concentrations will be compared to expected results and will be provided along with protocols for staining and recording of human iPSC-CMs with ElectroFluors™.

Potential Problems and Alternative Approaches. iPSC-CM cell cultures may show significant variability, which we plan to minimize using the only commercial source of cells approved for CiPA, the Fuji Cell Dynamics ICell\hat{ }2, in-house. If necessary, some experiments could also be done at an independent CRO. Since action potential duration is dependent on firing rate, entrainment using extracellular stimulation will be necessary to fix the firing rate for comparison purposes. Currently, platinum electrodes are available, but IonOptix carbon electrodes and/or ChRiff stimulation could be implemented if difficulties arise. We will look to pharma companies for advice in case other drugs not mentioned would be relevant to test, perhaps drugs with effects not predicted in previous CiPA-related studies using VSDs (Blinova et al., 2017, Kilfoil et al. 2021).

Metrics. Optical APs that reproduce drug concentration dependent APD changes, compared to patch clamp recordings.

### Overall Milestones

As new ElectroFluors™ are offered, all data from Aims 1 and 2 will be deposited on our website.

6 Months: Market 3 new ElectroFluors™ offering choices of enhanced solubility or persistence.
1 Year: First 3 chromene-based ElectroFluors™ offered in the catalog. Collaboration on ratiometric voltage AP recording following optogenetic activation (with Sacconi).
1.5 Years: Extend the optimal wavelength of chromene-based ElectroFluors™ beyond 730 nm. Publish combined ratiometric voltage and calcium imaging in beating iPSC-CM tissue.
2 Years: Full catalog of 15 ElectroFluors™ (10 beyond our current offering) spanning the visible and near IR spectrum and with a range of solubilities and persitence. Publish ratiometric voltage imaging with in-vivo pig in the near IR with chromene-based ElectroFluors™ (with Figueiras-Rama).

Features and advantages of this disclosure are apparent from the detailed specification, and the claims cover all such features and advantages. Numerous variations will occur to those skilled in the art, and any variations equivalent to those described in this disclosure fall within the scope of this disclosure. Those skilled in the art will appreciate that the conception upon which this disclosure is based may be used as a basis for designing other compositions and methods for carrying out the several purposes of this disclosure. As a result, the claims should not be considered as limited by the description or examples.

1. A compound having the structure: 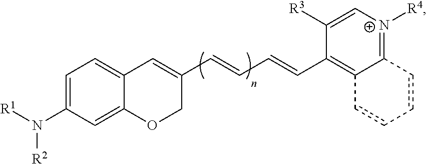
wherein R¹-R³ are independently hydrogen, halide, C₁-C₁₂ alkyl, C₁-C₁₂ heteroalkyl, C₂-C₁₂ alkenyl, C₂-C₁₂ alkynyl, C₁-C₁₂ haloalkyl, C₂-C₁₂ haloalkenyl, cycloalkyl, heterocycloalkyl, bicyclic cycloalkyl, bicyclic heterocycloalkyl, cycloalkenyl, cycloalkynyl, aryl, or heteroaryl;
wherein n is an integer ranging from 0-4;
wherein R⁴ is (C₁-C₁₂)—R⁵, wherein R⁵ is NR⁶⁻⁸, wherein R₆—R⁸ are independently C₁-C₁₂ alkyl; sulfonate; carboxylic acid; amino; —C(O)O—R⁹, wherein R⁹ is succinimidyl; or —NH—C(O)—R¹⁰—R¹¹, wherein R¹⁰ is C₁-C₄ alkyl and R¹¹ is maleimide; and
wherein the dashed line (- - - -) denotes an optional ring structure.

2. The compound of claim 1, wherein R¹ and R² are independently C₁-C₁₂ alkyl.

3. (canceled)

4. (canceled)

5. The compound of claim 1, wherein R¹ and R² are independently C₃-C₆ alkyl.

6. The compound of claim 1, wherein nis 0 or 1.

7. The compound of claim 1, wherein R³ is hydrogen or halide.

8. The compound of claim 1, wherein R³ is hydrogen or fluoride.

9. The compound of claim 1, wherein R⁴ has the structure:

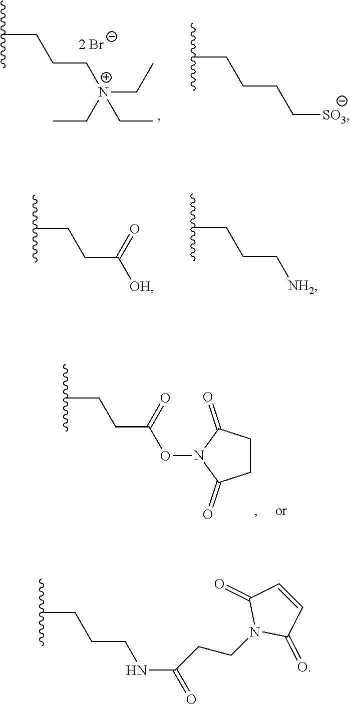

10. The compound of claim 1, which has the structure: 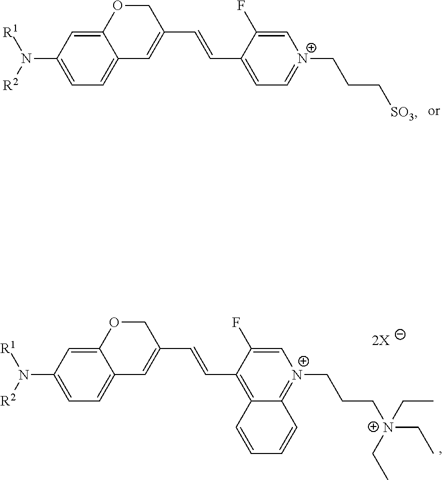
wherein X is a halide; and wherein R¹ and R² are independently n-butyl, n-pentyl, or n-hexyl.

11. The compound of claim 10, wherein X is Br.

12. A method of making a compound, the method comprising the steps of:
reacting a compound having a structure: 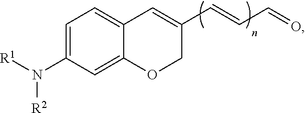
wherein R¹ and R² are independently hydrogen, halide, C₁-C₁₂ alkyl, C₁-C₁₂ heteroalkyl, C₂-C₁₂ alkenyl, C₂-C₁₂ alkynyl, C₁-C₁₂ haloalkyl, C₂-C₁₂ haloalkenyl, cycloalkyl, heterocycloalkyl, bicyclic cycloalkyl, bicyclic heterocycloalkyl, cycloalkenyl, cycloalkynyl, aryl, or heteroaryl, and
wherein n is an integer ranging from 0-4;
with a compound having a structure: 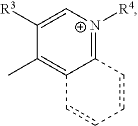
wherein R³ is hydrogen, halide, C₁-C₁₂ alkyl, C₁-C₁₂ heteroalkyl, C₂-C₁₂ alkenyl, C₂-C₁₂ alkynyl, C₁-C₁₂ haloalkyl, C₂-C₁₂ haloalkenyl, cycloalkyl, heterocycloalkyl, bicyclic cycloalkyl, bicyclic heterocycloalkyl, cycloalkenyl, cycloalkynyl, aryl, or heteroaryl;
wherein R⁴ is (C₁-C₁₂)—R⁵, wherein R⁵ is NR⁶⁻⁸, wherein R⁶-R⁸ are independently C₁-C₁₂ alkyl; sulfonate; carboxylic acid; amino; —C(O)O—R⁹, wherein R⁹ is succinimidyl; or —NH—C(O)—R¹⁰-R¹¹, wherein R¹⁰ is C₁-C₄ alkyl and R¹¹ is maleimide; and
wherein the dashed line (- - - -) denotes an optional ring structure.
in a condensation reaction to form a compound having a structure:

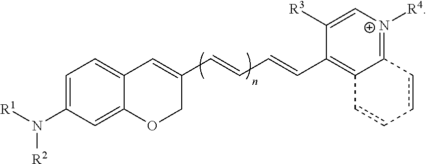

13. (canceled)

14. (canceled)

15. (canceled)

16. A method of making a compound, the method comprising the steps of:
propargylation of a compound having the structure: 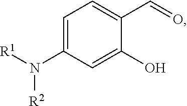
wherein R¹ and R² are independently hydrogen, halide, C₁-C₁₂ alkyl, C₁-C₁₂ heteroalkyl, C₂-C₁₂ alkenyl, C₂-C₁₂ alkynyl, C₁-C₁₂ haloalkyl, C₂-C₁₂ haloalkenyl, cycloalkyl, heterocycloalkyl, bicyclic cycloalkyl, bicyclic heterocycloalkyl, cycloalkenyl, cycloalkynyl, aryl, or heteroaryl,
into a compound having the structure: 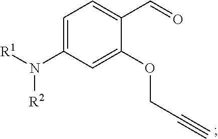
cyclization in the presence of malononitrile to form a compound having the structure: 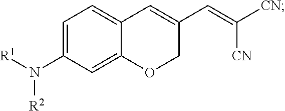
hydrolysis to form a compound having the structure: 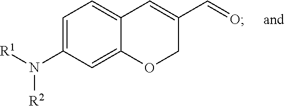
performing an olefination reaction, from 1 to 5 times, to form a compound having the structure: 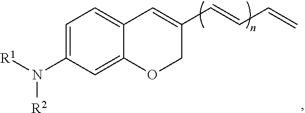
wherein n is an integer ranging from 0-4.

17. The method of claim 16, wherein 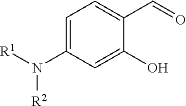
is prepared by formylation of a compound having the structure:

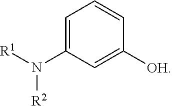

18. The method of claim 17, wherein 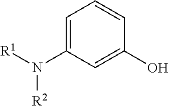
is prepared by alkylation of a compound having the structure:

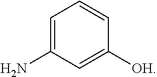

19. The method of claim 16, wherein the olefination reaction is a Wittig reaction.

20. The method of claim 16, wherein n is 0.

21. (canceled)

22. (canceled)

23. (canceled)

24. (canceled)

25. A composition comprising the compound of claim 1.

26. The composition of claim 25, which further comprises a desicant.

27. The composition of claim 25, which further comprises a solvent capable of at least partially dissolving the compound.

28. The composition of claim 27, which is in kit form comprising one or more packages having the compound therein.

29. The composition of claim 27, which is in kit form comprising one or more packages, wherein the compound and the solvent are packaged separately.

30-37. (canceled)
